(19) United States
(12) Patent Application Publication
(10) Pub. No.: US 2023/0210768 A1
(43) Pub. Date: Jul. 6, 2023

(54) AN INHALED IL-1 BLOCKADE TREATMENT FOR RESPIRATORY TRACT IMMUNOPATHOLOGY

(71) Applicant: Altavant Sciences GmbH, Basel (CH)

(72) Inventors: Stephen A. WRING, South Boston, VA (US); Lyn A. BARANOWSKI, Brooklyn, NY (US); Katelyn R. CRIZER, Chapel Hill, NC (US); Michelle PALACIOS, Garner, NC (US)

(21) Appl. No.: 17/926,297

(22) PCT Filed: May 20, 2021
(86) PCT No.: PCT/IB2021/000347

§ 371 Date: Nov. 18, 2022

Related U.S. Application Data
(60) Provisional application No. 63/072,895, filed on Aug. 31, 2020.
(60) Provisional application No. 63/028,494, filed on May 21, 2020.

(51) Int. Cl. A61K 9/00; A61K 38/17

(52) U.S. Cl. CPC A61K 9/0078; A61K 38/1793

(57) ABSTRACT

The invention is directed to a method for treating an inflammatory disorder of the lower airways in a human subject in need thereof, comprising administering an effective amount of a recombinant human IL-1 receptor antagonist (rhIL-IRa) directly to the lower airways in the human subject; wherein the inflammatory disorder is caused by a coronavirus infection.

This application claims the benefit of and priority to U.S. Ser. No. 63/028,494 filed May 21, 2020 and U.S. Ser. No. 63/072,895 filed Aug. 31, 2020, the contents of each of which are hereby incorporated by reference in their entireties.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals for treating various lower airways disorders.

### BACKGROUND

Development of SARS in patients is associated with exuberant over-production of pro-inflammatory cytokines that drive a hyper-immune inflammatory response. See Mehta, P. et al., COVID-19: consider cytokine storm syndromes and immunosuppression, The Lancet, Vol. 395 (March 2020) pp. 1033-1034. These aberrant cytokine profiles notably include marked elevations in IL-1 and IL-6. See Conti, P., et al. Introduction of pro-inflammatory cytokines (IL-1 and IL-6) and lung inflammation by COVID-19: anti-inflammatory strategies, Journal of Biological Regulators and Homeostatic Agents, Vol. 24, no. 2 (March 2020), pp. 11-15; Huang, C., et al. Clinical features of patients infected with 2019 novel coronavirus in Wuhan, China, The Lancet, Vol., 395 (February 2020), pp. 497-506; Shakoory, B., et al., Interleukin-1 receptor blockade is associated with reduced mortality in sepsis patients with features of the macrophage activation syndrome: Re-analysis of a prior Phase III trial, Crit. Care Med., Vol. 44, no. 2 (February 2016) pp. 275-281. It is thought this accentuated immune response and the broadly dysregulated cytokine storm (cytokine storm syndrome, CSS) occurs as an immunological response driven by activation of toll-like receptors (TLRs) by viral triggers. See Cron, R. et al. Don't Forget the Host: COVID-19 Cytokine Storm, The Rheumatologist, (March 2020).

Cytokine storm syndrome is a broad term that is associated with the clinical complications of Coronavirus disease 2019 (COVID-19) caused by the SARS-CoV-2 strain of coronaviruses. This cytokine response can ultimately lead to acute respiratory distress and if untreated multi-organ failure. See Cron, R. et al. Don't Forget the Host: COVID-19 Cytokine Storm, The Rheumatologist, (March 2020). An estimated 20% of individuals infected with COVID-19 require hospitalization with a subset of severely-infect patients who require intensive care. See Pan, F. et al. Time Courses of Lung Changes On Chest CT During Recovery From 2019 Novel Coronavirus (COVID-19) Pneumonia, Radiology, (2020). Early disease appears confined to the lower respiratory tract. The earlier therapy is implemented for CSS, particularly while disease is limited to respiratory tract, the better the likely outcomes. See Cron, R. et al. Don't Forget the Host: COVID-19 Cytokine Storm, The Rheumatologist, (March 2020). Therefore, there remains a need for pharmaceutical compositions and methods to treat respiratory tract immunopathology.

### SUMMARY OF THE INVENTION

In one aspect, the invention provides for a method for treating an inflammatory disorder of the lower airways in a human subject in need thereof, comprising administering an effective amount of a recombinant human IL-1 receptor antagonist (rhIL-1Ra) directly to the lower airways in the human subject, wherein the inflammatory disorder is caused by a coronavirus infection.

In some embodiments, the rhIL-1Ra is anakinra. In some embodiments, the anakinra is a component of a composition, and wherein the composition is an inhaled formulation. In some embodiments, the inhaled formulation is ALTA-2530. ALTA-2530 as described herein refers to an inhaled recombinant interleukin-1 alpha and beta (IL-1α and IL-1β) receptor antagonist protein. In some embodiments, the coronavirus infection is caused by a coronavirus selected from the group consisting of SARS-CoV-2, SARS-CoV, MERS-CoV, 229E, NL63, OC43, and HKU1. In some embodiments, the coronavirus infection is caused by a coronavirus selected from the group consisting of SARS-CoV-2 and a mutant thereof. In some embodiments, the SARS-CoV-2 mutant is a variant selected from the group consisting of B.1.526, B.1.526.1, B.1.525, B.1.617, B.1.617.1, B.1.617.2, B.1.617.3, B.1.1.7, B.1.351, B.1.427, B.1.429, P.1, and P.2. In some embodiments, the human subject is diagnosed with COVID-19. In some embodiments, the inflammatory disorder of the lower airways is acute respiratory distress syndrome or cytokine storm syndrome. In some embodiments, the rhIL-1Ra is nebulized. In some embodiments, the nebulized rhIL-1Ra has a mass median aerodynamic diameter (MMAD) of about 1 μm to 15 μm. In some embodiments, the nebulized rhIL-1Ra has a mass median aerodynamic diameter (MMAD) of about 3 μm. In some embodiments, the nebulized rhIL-1Ra is delivered using a nebulizer. In some embodiments, the nebulizer is selected from the group consisting of PARI eFlow nebulizer, PARI VELOX nebulizer, Philips iNeb Advanced nebulizer, Philips InnoSpire Go nebulizer, a Vectura nebulizer, and a Monaghan Medical AeroEclipse II nebulizer. In some embodiments, the nebulizer is a PARI nebulizer or a Vectura nebulizer. In some embodiments, the rhIL-1Ra inhibits at least one pro-inflammatory cytokine selected from the group consisting of interleukin 1 alpha (IL-1α), interleukin 1 beta (IL-1β), interleukin 6 (IL-6), tumor necrosis factor alpha (TNFα), and interleukin 18 (IL-18).

In another aspect, the invention provides for a method for treating an inflammatory disorder of the lower airways in a human subject in need thereof, comprising administering an effective amount of a recombinant human IL-1 receptor antagonist (rhIL-1Ra) directly to the lower airways in the human subject, wherein the rhIL-1Ra causes blockade of interleukin 1 to about the same degree as caused by the upregulation of endogenous IL-1Ra during a restoration of physiologic immune regulation.

In some embodiments, the rhIL-1Ra is anakinra. In some embodiments, the anakinra is a component of a composition, and wherein the composition is an inhaled formulation. In some embodiments, the inhaled formulation is ALTA-2530. In some embodiments, the inflammatory disorder is caused by a coronavirus infection. In some embodiments, the human subject is diagnosed with a coronavirus infection. In some embodiments, the coronavirus infection is COVID-19. In some embodiments, the coronavirus infection is caused by a coronavirus selected from the group consisting of SARS-CoV-2, SARS-CoV, MERS-CoV, 229E, NL63, OC43, and HKU1. In some embodiments, the coronavirus infection is caused by a coronavirus selected from the group consisting of SARS-CoV-2 and a mutant thereof. In some embodiments, the SARS-CoV-2 mutant is a variant selected from the group consisting of B.1.526, B.1.526.1, B.1.525, B.1.617, B.1.617.1, B.1.617.2, B.1.617.3, B.1.1.7, B.1.351, B.1.427, B.1.429, P.1, and P.2. In some embodiments, the inflammatory disorder of the lower airways is acute respiratory distress syndrome or cytokine storm syndrome. In some embodiments, the rhIL-1Ra is nebulized. In some embodiments, the nebulized rhIL-1Ra has a mass median aerodynamic diameter (MMAD) of about 1 μm to 15 μm. In some embodiments, the nebulized rhIL-1Ra has a mass median aerodynamic diameter (MMAD) of about 3 μm. In some embodiments, the nebulized rhIL-1Ra is delivered using a nebulizer. In some embodiments, the nebulizer is selected from the group consisting of PARI eFlow nebulizer, PARI VELOX nebulizer, Philips iNeb Advanced nebulizer, Philips InnoSpire Go nebulizer, a Vectura nebulizer, and AeroEclipse II nebulizer. In some embodiments, the nebulizer is a PARI nebulizer or a Vectura nebulizer. In some embodiments, the rhIL-1Ra inhibits at least one pro-inflammatory cytokine selected from the group consisting of interleukin 1 alpha (IL-1α), interleukin 1 beta (IL-1β), interleukin 6 (IL-6), tumor necrosis factor alpha (TNFα), and interleukin 18 (IL-18).

### BRIEF DESCRIPTION OF THE DRAWINGS

The following figures depict illustrative embodiments of the invention.

FIG. 1 is a schematic diagram showing the mechanism of action of acute inflammation caused by COVID-19 infection and the mechanism of action of ALTA-2530 to reduce inflammation, according one or more embodiments disclosed herein.

FIG. 2 shows concentration versus time profiles for rhIL-1Ra in ELF and serum following single doses to rat, according one or more embodiments disclosed herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The term “lower airways” or “lower respiratory tract” when used herein refers to or describes anatomic regions below the larynx including the trachea and lungs, as well as lower regions of the lung.

The terms “treating,” “treatment,” and “therapy” as used herein refer to attempted reduction or amelioration of the progression, severity and/or duration of a disorder, or the attempted amelioration of one or more symptoms thereof resulting from the administration of one or more modalities (e.g., one or more therapeutic agents such as a compound or composition of the invention).

As used herein, “therapeutically effective amount” or “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the terms “subject” and “patient” are used interchangeably herein. The terms “subject” and “subjects” refer to an animal, preferably a mammal including a nonprimate and a primate (e.g., a monkey such as a cynomolgus monkey, a chimpanzee, and a human), and more preferably a human. The term “animal” also includes, but is not limited to, companion animals such as cats and dogs; zoo animals; wild animals; farm or sport animals such as ruminants, non-ruminants, livestock and fowl (e.g., horses, cattle, sheep, pigs, turkeys, ducks, and chickens); and laboratory animals, such as rodents (e.g., mice, rats), rabbits; and guinea pigs, as well as animals that are cloned or modified, either genetically or otherwise (e.g., transgenic animals).

As used herein, “a” or “an” means at least one, unless clearly indicated otherwise. The term “about,” unless otherwise indicated, refers to a value that is no more than 10% above or below the value being modified by the term. For example, the term “about 5% (w/w)” means a range of from 4.5% (w/w) to 5.5% (w/w).

As used herein, unless indicated otherwise, the terms “composition” and “composition of the invention”, are used interchangeably. Unless stated otherwise, the terms are meant to encompass, and are not limited to, pharmaceutical compositions and nutraceutical compositions containing drug substance (e.g., anakinra). The composition may also contain one or more “excipients” that are inactive ingredients or compounds devoid of pharmacological activity or other direct effect in the diagnosis, cure, mitigation, treatment, or prevention of disease or to affect the structure or any function of the human.

### DETAILED DESCRIPTION

Unchecked, excessive cytokine release can lead to acute respiratory distress syndrome and cytokine storm syndrome leading to multi-organ failure. Disclosed herein are compositions and method of treating the potentially severe respiratory tract damage that may occur particularly to lower regions of the lung, following viral infections caused by SARS-CoV-2, SARS-CoV, MERS-CoV, 229E, NL63, OC43, and HKU1. In some embodiments, the viral infection is caused by SARS-CoV-2 or a mutant thereof, and wherein the SARS-CoV-2 mutant is a variant such as B.1.526, B.1.526.1, B.1.525, B.1.617, B.1.617.1, B.1.617.2, B.1.617.3, B.1.1.7, B.1.351, B.1.427, B.1.429, P.1, or P.2. In some embodiments, the treatment comprises delivery of oral inhaled nebulized recombinant human Interleukin-1 receptor antagonist (rhIL-1Ra) to dampen or reverse the local hyper-inflammatory response and tissue damage caused following inappropriately high local cytokine release. In some embodiments, the recombinant human Interleukin-1 receptor antagonist (rhIL-1Ra) is anakinra.

In some embodiments, an inhaled formulation of a recombinant human IL-1 receptor antagonist (rhIL-1Ra) is administered to treat severe acute respiratory syndrome (SARS) that may develop in patients following viral infections, such as by coronavirus.

In some embodiments, a pharmaceutical composition includes a combination of a small volume novel formulation of rhIL-1Ra that can be delivered to lungs to achieve higher respiratory tract levels of IL-1Ra than feasible with SC or IV treatment unless high-dose continual IV infusion is employed.

In some embodiments, an inhaled recombinant interleukin-1 alpha and beta (IL-1α and IL-1β) receptor antagonist protein (rhIL-1Ra; also known as “ALTA-2530”) is used for the treatment of bronchiolitis obliterans syndrome (BOS) in post-lung transplant patients. Bronchiolitis obliterans syndrome is characterized by increased IL-1 production and down-stream over-activation of the innate immune response in lung tissue. Inhaled IL1-RA successfully mitigated BOS slowing disease progression in 3 patients with late stage disease.

Without intending to be bound by any theory, IL-1 inhibition by ALTA-2530 blocks the innate immune system as represented by the NLRP3 inflammasome, toll-like receptors and Caspase 1 [Cron, R. IL-1 Family Blockade in Cytokine Storm Syndromes, In Cytokine Storm Syndrome (pp. 549-559). Springer, Cham. (2019)]. By inhibiting IL-1, ALTA-2530 is the most upstream block of the cytokines of the innate system, including IL-6, TNFα and IL-18. Therefore, inhibition of IL-1 may be the optimal approach to inhibit key elements of the innate immune system that drive the development of CSS.

Subcutaneous and intravenous treatment with rhIL-1Ra has been evaluated for treatment of macrophage activation syndrome, a sub-set of CSS, in patients where standard of care was failing. Subcutaneous doses of anakinra (up to 4× the approved dose for RA) were likewise associated with worsening condition. Intravenous rhIL-1Rs infusion were initiated and doses ramped to 1.5-2 mg/kg/hr (2400 mg/day). At this dose 80% (4/5) of patients responded with rapid clinical improvement. See Shakoory, B., et al., Interleukin-1 receptor blockade is associated with reduced mortality in sepsis patients with features of the macrophage activation syndrome: Re-analysis of a prior Phase III trial, Crit. Care Med., Vol. 44, no. 2 (February 2016) pp. 275-281. However, high doses of rhIL-1RA were associated with renal damage, increases in hepatic transaminases and cytopenia (leukopenia). The approved subcutaneous dose for rhIL-1Ra (anakinra, a SC product) is 100 mg/day.

Similarly, in a re-analysis of a Phase III clinical trial in patients with MAS associated with sepsis IV infusion of rhIL-1RA (2 mg/kg/hr) yielded a significant improvement in 28-day survival (47% reduction in mortality). See Shakoory, B., et al., Interleukin-1 receptor blockade is associated with reduced mortality in sepsis patients with features of the macrophage activation syndrome: Re-analysis of a prior Phase III trial, Crit. Care Med., Vol. 44, no. 2 (February 2016) pp. 275-281.

In COVID-19 a retrospective cohort study high-dose IV rhIL-1Ra administered to patients managed with non-invasive ventilation outside of ICU achieved clinical improvement in 72% (21/29) patients. Patients received either background therapy (SOC) alone or with anakinra (5 mg/kg IV BID or 100 mg SC BID). Low SC doses did not demonstrate clinical improvement after 7 days of treatment. At day 21 survival of patients on high dose IV anakinra was 72% (21/29) and 50% (6/12) in those receiving SOC. See Cavalli, G. et al. Interleukin-1 blockade with high-dose anakinra in patients with COVID-19, acute respiratory distress syndrome, and hyperinflammation: a retrospective cohort study, The Lancet (May 2020) pp. 1-7.

A likely challenge with parenteral delivery of rhIL-1RA is limited tissue distribution. Indeed, the approved subcutaneous (SC) route achieves only sub-optimal levels in lung tissue (2% of delivered dose).

In some embodiments, an rhIL-1Ra may be nebulized and achieve particles mass median aerodynamic diameter (MMAD) of about between about 1 μm and about 5 μm, between about 5 μm and between about 10 μm, between about 10 μm and 15 μm, or between about 15 μm and 20 μm. Preferably, the MMAD is about 3 μm, consistent with delivery to lower regions of the lung. An MMAD of about 3 μm permits a high delivered dose to the region of the respiratory tract most associated with COVID-19 CSS. Further, inhaled delivery lowers systemic exposure with the potential benefit of reducing the risk of the adverse events associated with high dose IV infusion therapy.

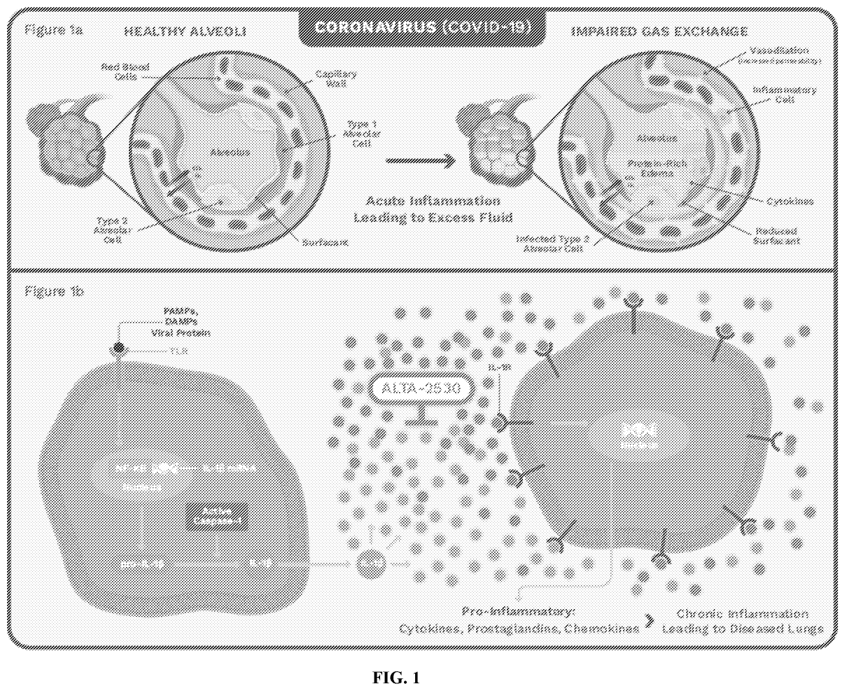

Disclosed herein is an inhaled IL-1 receptor antagonist, ALTA-2530, that can be used to attenuate inflammation and acute lung injury associated with Acute Respiratory Distress Syndrome (ARDS). ARDS is a frequent life-threatening complication of COVID-19 and a leading cause of death. As shown in FIG. 1, development of ARDS is associated with cytokine storm syndrome (CSS) involving exuberant overproduction of proinflammatory cytokines that leads to a hyper-inflammatory state and associated lung tissue injury, alveolar edema and impaired oxygen transfer.

The cytokine IL-1β is an agonist that binds to the IL-1 receptor (IL-1R1) to drive activation of the innate immune system and the inflammatory cascade derivative of activation of toll-like receptors, the NLRP3 inflammasome, and Caspase 1. Increased plasma IL-1b (>400 pg/mL) during the first week of ARDS has been proposed as predictive of poorer clinical outcome. As further shown in FIG. 1, by inhibiting IL-1 signaling (IL-1 blockade), ALTA-2530, an inhaled formulation of recombinant human IL-1 receptor antagonist (rhIL-1Ra, anakinra), may block the increased cytokine expression characteristic of CSS, including IL-6, TNFα and IL-18.

Importantly, IL-1 blockade contributes to the physiologic regulation of inflammation, and endogenous IL-1Ra is upregulated in response to IL-1 to limit the inflammatory response. Thus, pharmaceutical IL-1 blockade is not only an effective and targeted potential therapy, but its mechanism of action may be considered akin to the restoration of physiologic immune regulation.

Anakinra, a subcutaneous formulation of rhIL-1Ra, is approved for rheumatoid arthritis and cryopyrin-associated periodic syndromes (CAPS) (Kineret™). IL-1Ra binds to the IL-1RI receptor with comparable avidity as IL-1β and the competitive nature of binding necessitates maintaining pharmacologically relevant levels in lung tissue for COVID-19 (see FIG. 1). High-dose intravenous anakinra has reduced mortality in COVID-19 and macrophage activation syndrome but can lead to kidney injury and leukopenia increasing risk of treatment-related complications, particularly in patients with co-morbidities. High IV doses of Anakinra are likely required owing to limited tissue distribution to the lung (in nonclinical studies only ˜2% of a SC dose distributed to lung).

ALTA-2530 is a sequence-identical protein to anakinra that has been reformulated for pulmonary delivery to achieve higher alveolar levels of IL-1Ra than feasible with SC or IV treatment. Our nonclinical studies with ALTA-2530 have shown delivery as a nebulized solution to the lung can reduce the daily dose, and lower systemic exposure thereby reducing risk of renal damage and leukopenia.

ALTA-2530 drug substance is manufactured at kilo scale. The formulated drug product has been optimized to deliver particle mass median aerodynamic diameters (MMAD) of ˜3 μm, consistent with delivery to the small airways of the distal regions of lung—the site of early inflammation and damage. Impurity profiling by HPLC-UV and HPLC-SEC methods demonstrated rhIL-1Ra protein was stable during nebulization. Full biological activity was retained as assessed by an in vitro cell-based assay.

In some embodiments, ALTA-2530 is compatible with several 510k approved or CE accredited hand-held and/or ventilator compatible nebulizers thereby making it convenient for ambulatory COVID-19 patients as well as those requiring mechanical ventilation.

ALTA-2530 represents a safe molecule with a well-defined mechanism of action, evidence of clinical efficacy following IV therapy in COVID-19, kg scale production, and compatibility with regulatory agency approved nebulizers.

### Compositions of IL-1 Receptor Antagonists

In one aspect, a pharmaceutical composition is described, including an interleukin-1 receptor antagonist and one or more additional components each selected from the group consisting of a buffer, a stabilizer, and a tonicity modifier.

In some embodiments, the interleukin-1 receptor antagonist is anakinra. Other interleukin-1 receptor antagonists are contemplated.

In some embodiments, the buffer is selected from the group consisting of citrate, phosphate, succinate, histidine, glutamate, pyrophosphate, 4-(2-hydroxyethyl)-1-piperazineethanesulfonic acid (HEPES), and a combination thereof. In some embodiments, the pharmaceutical composition is a liquid composition comprising citrate in a concertation of between about 0.5 mM and 20 mM.

In some embodiments, the concentration of citrate is about 20 mM. In some embodiments, the pharmaceutical composition is a liquid composition comprising phosphate in a concentration of between about 1 mM and 50 mM, or about 10 mM.

In some embodiments, the pharmaceutical composition is a liquid composition comprising histidine in a concentration of between about 5 mM and 50 mM or about 10 mM.

In some embodiments, the pharmaceutical composition is a liquid composition comprising glutamate in a concentration of between about 1 mM and 50 mM. In some embodiments, the pharmaceutical composition is a liquid composition comprising pyrophosphate in a concentration of between about 1 mM and 50 mM.

In some embodiments, the pharmaceutical composition is a liquid composition comprising 4-(2-hydroxyethyl)-1-piperazineethanesulfonic acid (HEPES) in a concentration of between about 10 mM and 50 mM or about 10 mM.

In some embodiments, the stabilizer is selected from the group consisting of a surfactant, a chelating agent, a sugar, and a combination thereof. In some embodiments, the surfactant is selected from the group consisting of polysorbate 80, polysorbate 20, polyoxyethylene (23) lauryl ether (Brij™ 35), sorbitan trioleate (Span™ 85), and a combination thereof.

In some embodiments, the pharmaceutical composition is a liquid composition comprising polysorbate 80 in a concentration of between about 0.01% and 1% (w/v) or about 0.1% (w/v).

In some embodiments, the pharmaceutical composition is a liquid composition comprising polysorbate 20 in a concentration of between about 0.00001% and 1% (w/v), or between about 0.00001% and 0.01% (w/v). In any one of the embodiments described herein, the pharmaceutical composition is a liquid composition comprising polysorbate 20 in a concentration of about 0.00001% (w/v), 0.0001% (w/v), or 0.001% (w/v).

In some embodiments, the pharmaceutical composition is a liquid composition comprising polyoxyethylene (23) lauryl ether (Brij™ 35) in a concentration of between about 0.00001% and 0.01% (w/v). In some embodiments, the pharmaceutical composition is a liquid composition comprising sorbitan trioleate (Span™ 85) in a concentration of between about 0.1% and 5.0% (w/v), about 0.8 (w/v), 0.85 (w/v), or 0.86% (w/v).

In some embodiments, the chelating agent is ethylenediaminetetraacetic acid (EDTA) disodium. In some embodiments, the pharmaceutical composition is a liquid composition comprising ethylenediaminetetraacetic acid (EDTA) disodium in a concentration of between about 0.05 mM and 1 mM or about 0.5 mM.

In some embodiments, the sugar is selected from the group consisting of trehalose, sucrose, glycerol, sorbitol, and a combination thereof. In some embodiments, the pharmaceutical composition is a liquid composition and the concentration of the sugar is greater than about 40% (w/v).

In some embodiments, the tonicity modifier is selected from the group consisting of sodium chloride, mannitol, taurine, hydroxyproline, proline, and a combination thereof. In some embodiments, the pharmaceutical composition is a liquid composition comprising sodium chloride in a concentration of between about 120 mM and 180 mM or about 140 mM.

In some embodiments, the pharmaceutical composition is a liquid composition comprising mannitol in a concentration of between about 5 mg/mL and 50 mg/mL or about 10 mg/mL.

In some embodiments, the pharmaceutical composition is a liquid composition comprising taurine in a concentration of between about 15 mg/mL and 50 mg/mL or about 30 mg/mL.

In some embodiments, the pharmaceutical composition is a liquid composition comprising hydroxyproline in a concentration of between about 15 mg/mL and 50 mg/mL or about 26 mg/mL.

In some embodiments, the additional components comprise citrate, ethylenediaminetetraacetic acid (EDTA) disodium, polysorbate 80, and sodium chloride. In some embodiments, the additional components comprise phosphate, ethylenediaminetetraacetic acid (EDTA) disodium, polysorbate 80, and sodium chloride.

In some embodiments, the additional components comprise phosphate, mannitol, ethylenediaminetetraacetic acid (EDTA) disodium, and sodium chloride. In some embodiments, the additional components comprise phosphate, mannitol, ethylenediaminetetraacetic acid (EDTA) disodium, polysorbate 80, and sodium chloride.

In some embodiments, the additional components comprise phosphate, mannitol, ethylenediaminetetraacetic acid (EDTA) disodium, polysorbate 20, and sodium chloride. In some embodiments, the additional components comprise phosphate, mannitol, ethylenediaminetetraacetic acid (EDTA) disodium, sorbitan trioleate (Span™ 85), and sodium chloride.

In some embodiments, the additional components comprise phosphate, trehalose, ethylenediaminetetraacetic acid (EDTA) disodium, polysorbate 80, and sodium chloride. In some embodiments, the additional components comprise phosphate, sucrose, ethylenediaminetetraacetic acid (EDTA) disodium, polysorbate 80, and sodium chloride.

In some embodiments, the additional components comprise phosphate, ethylenediaminetetraacetic acid (EDTA) disodium, a tonicity modifier, and sodium chloride. In some embodiments, the additional components comprise phosphate, mannitol, ethylenediaminetetraacetic acid (EDTA) disodium, polysorbate 80, a tonicity modifier, and sodium chloride.

In some embodiments, the additional components comprise phosphate, trehalose, ethylenediaminetetraacetic acid (EDTA) disodium, polysorbate 20, a tonicity modifier, and sodium chloride. In some embodiments, the additional components comprise phosphate, sucrose, ethylenediaminetetraacetic acid (EDTA) disodium, sorbitan trioleate (Span™ 85), a tonicity modifier, and sodium chloride.

In some embodiments, the additional components comprise phosphate, a tonicity modifier, and sodium chloride. In some embodiments, the tonicity modifier is selected from the group consisting of taurine, hydroxyproline, and a combination thereof.

In some embodiments, the additional components comprise citrate, phosphate, ethylenediaminetetraacetic acid (EDTA) disodium, polysorbate 80, and sodium chloride. In some embodiments, the additional components comprise glutamate, ethylenediaminetetraacetic acid (EDTA) disodium, polysorbate 80, and sodium chloride.

In some embodiments, the additional components comprise citrate, trehalose, ethylenediaminetetraacetic acid (EDTA) disodium, polysorbate 80, and sodium chloride.

In some embodiments, the additional components comprise glutamate, mannitol, ethylenediaminetetraacetic acid (EDTA) disodium, polysorbate 80, and sodium chloride. In some embodiments, the additional components comprise phosphate, mannitol, and sodium chloride.

In some embodiments, the pharmaceutical composition is a liquid composition. In some embodiments, the pharmaceutical composition is a solid composition.

In some embodiments, the solid composition is a lyophilisate. In some embodiments, the pharmaceutical composition is reconstituted from a lyophilisate.

In another aspect, a kit is disclosed, including a pharmaceutical composition according to any one of embodiments described herein and a delivery device suitable for direct administration of the pharmaceutical composition to the respiratory tract of a patient.

In some embodiments, the respiratory tract comprises the lower or upper airways.

In some embodiments, the delivery device is configured to deliver an effective amount of the pharmaceutical composition via inhalation. In some embodiments, the delivery device is configured to deliver an effective amount of the pharmaceutical composition via direct instillation.

In some embodiments, the delivery device is selected from the group consisting of a nebulizer, an inhaler, and an aerolizer. In some embodiments, the delivery device is selected from the group consisting of a jet nebulizer, an ultrasonic nebulizer, a metered dose inhaler, and a dry powder inhaler. In some embodiments, the nebulizer is selected from the group consisting of the Philips iNeb Advanced nebulizer, the Philips InnoSpire Go nebulizer, the AeroEclipse II jet nebulizer, and the Aerogen Solo VM nebulizer.

In some embodiments, the pharmaceutical composition is a solution for nebulization delivered using a nebulizer such as the PARI eFlow nebulizer, the PARI VELOX nebulizer, the Philips iNeb Advanced nebulizer, the Philips InnoSpire Go vibrating mesh (VM) nebulizer, a Vectura nebulizer (e.g., FOX® vibrating mesh nebulizer; AKITA® JET device), a preclinical nebulizer (e.g., Aerogen Solo VM nebulizer), or any other suitable vibrating mesh or jet nebulizer. In some embodiments, the pharmaceutical composition is a solution for nebulization delivered using a PARI nebulizer. In some embodiments, the pharmaceutical composition is a solution for nebulization delivered using the Philips iNeb Advanced nebulizer or the Philips InnoSpire Go vibrating mesh (VM) nebulizer. In some embodiments, the pharmaceutical composition is a solution for nebulization delivered using a Vectura nebulizer (e.g., FOX® vibrating mesh nebulizer; AKITA® JET device). In some embodiments, the pharmaceutical composition is a solution for nebulization delivered using any suitable vibrating mesh or jet nebulizer. In some embodiments, the pharmaceutical composition is an extemporaneously prepared solution formulation for nebulization that can be produced at the preclinical and clinical study sites and is stable for nebulization over the dosing period and a minimum in-use period of 24 hours. In some embodiments, the pharmaceutical composition is a solution for nebulization stored at refrigeration temperatures. In some embodiments, the pharmaceutical composition developed for GLP toxicology and GMP clinical studies will preferably be the same or comparable to avoid any bridging studies (e.g., excipients will not differ, and ratios will not exceed GLP qualification levels). In some embodiments, the pharmaceutical composition's impurity profiles of the nebulized GMP clinical formulation will be similar to and will not exceed the impurity limits qualified in the GLP preclinical studies. In some embodiments, the pharmaceutical composition is a clinical formulation solution having concentration(s) suited to deliver 10-40 mg from the nebulizer (expressed as drug charge to nebulizer) in less than 5 minutes and ideally within 2-3 minutes using, for example, the PART eFlow, the PART VELOX nebulizer, the Philips iNeb Advanced nebulizer, the Philips InnoSpire Go nebulizer, or any other suitable vibrating mesh or jet nebulizer. In some embodiments, the pharmaceutical composition is reproducibly delivered, and pulmonary lung dose supports the clinical programs as demonstrated by chemical and aerosol performance stability over the in-use period and anticipated dosing duration. In some embodiments, the pharmaceutical composition has tolerability similar to or greater than thresholds qualified in preclinical freeze/thaw studies. In some embodiments, the pharmaceutical composition is stable based on preclinical stress stability studies. In some embodiments, the pharmaceutical composition meets purity standards based on preclinical filter compatibility studies. In some embodiments, the pharmaceutical composition does not exceed loss of content thresholds based on filter compatibility preclinical studies. In some embodiments, the pharmaceutical composition's stability of the nebulized GMP clinical formulation is similar to or greater than the stability thresholds qualified in preclinical studies. In some embodiments, the pharmaceutical composition's in-use period of the nebulized GMP clinical formulation is similar to the in-use period qualified in preclinical studies. In some embodiments, the pharmaceutical composition's storage conditions of the nebulized GMP clinical formulation is similar to the storage conditions qualified in preclinical studies. In some embodiments, the pharmaceutical composition's pH, osmolality, and appearance are similar to measures qualified in preclinical studies. In some embodiments, a protein concentration of the pharmaceutical composition is similar to a concentration qualified in preclinical studies. In some embodiments, purity of the pharmaceutical composition is similar to measures qualified in RPHPLC, SE-HPLC, reduced and non-reduced CE-SDS, and IEX-HPLC preclinical studies. In some embodiments, the levels of foreign and particulate matter, and subvisible particles in the pharmaceutical composition are similar to levels qualified in preclinical studies. In some embodiments, the levels of foreign and particulate matter, and subvisible particles in the pharmaceutical composition are similar to levels qualified in preclinical studies. In some embodiments, the pharmaceutical composition's aerosol particle size distribution by NGI of the nebulized GMP clinical formulation will be similar to the particle size distribution listed in USP 601. In some embodiments, the pharmaceutical composition's delivered dose using breath simulator will be similar to the dose listed in USP 1601 and USP 601 over the entire duration of dosing. In some embodiments, the pharmaceutical composition's potency will be similar to potency qualified in preclinical cell-based bioassay studies. In some embodiments, the pharmaceutical composition's measure of circular dichroism, viscosity, surface tension, formulation density, droplet size and distribution (e.g., as measured by Malvern Spraytec or equivalent), dynamic light scattering (DLS), and turbidity will be similar to measures qualified in preclinical studies.

In some embodiments, the pharmaceutical composition is a liquid composition and the delivery device is configured to deliver the liquid composition. In some embodiments, the pH of the liquid composition is between about 5 and 8.

In some embodiments, the osmolality of the liquid composition is between about 200 mOsm/kg and 400 mOsm/kg. In some embodiments, the osmolality is about 300 mOsm/kg.

In some embodiments, the droplet size of the liquid composition produced by the delivery device is between about 0.5 μm and 10 μm in diameter. In some embodiments, the droplet size of the liquid composition produced by the delivery device is suitable for preferentially targeting the lower airways.

In some embodiments, the droplet size of the liquid composition produced by the delivery device is between about 5 μm and 50 μm in diameter. In some embodiments, the droplet size of the liquid composition produced by the delivery device is suitable for preferentially targeting the upper airways. In some embodiments, the conductivity of the liquid composition is less than 2.5 μS/cm.

In some embodiments, the pharmaceutical composition is a solid composition and the delivery device is configured to deliver the solid composition. In some embodiments, the solid composition comprises particles having a mass median aerodynamic diameter (MMAD) between about 0.1 μm and 20 μm. In some embodiments, the MMAD of the particles is less than about 5 μm. In some embodiments, the MMAD of the particles is less than about 4.5, 4.0, 3.5, 3.0, 2.5, 2.0, 1.5, 1.0 or 0.5 μm, or the MMAD of the particles is in a range bound by any two numbers disclosed herein. In some embodiments, the MMAD of the particles is between about 2.0-5.0, 2.0-4.0, 2.0-3.0, 2.10-3.50, or 2.10-3.20 μm. In some embodiments, the MMAD of the particles is less than about 4 μm. In some embodiments, the MMAD of the particles is from about 2.5 to about 4 μm. In some embodiments, the MMAD of the particles is less than about 3.5 μm.

In some embodiments, the solid composition comprises particles having a mass median diameter (MMD) between about 0.1 μm and 20 μm. In some embodiments, the solid composition comprises particles having a mass median aerodynamic diameter (MMAD) between about 1 μm and 5 μm and a mass median diameter (MMD) between about 5 μm and 30 μm. In some embodiments, the ratio of MMD to MMAD is between about 2 and 30. In some embodiments, the ratio of MMD to MMAD is between about 5 and 30.

In some embodiments, the solid composition has a tap density of less than about 1 g/cm³. In some embodiments, the solid composition has a rugosity between about 1 and 6.

In some embodiments, the solid composition comprises porous particles. In some embodiments, the solid composition comprises swellable particles.

In some embodiments, the porous particles comprise biodegradable polymers. In some embodiments, the solid composition further comprises a salt of a fatty acid or a derivative thereof.

In some embodiments, the salt is selected from the group consisting of magnesium stearate, sodium stearyl fumarate, sodium stearyl lactylate, sodium lauryl sulfate, magnesium lauryl sulfate, and a combination thereof. In some embodiments, the solid composition comprises particles having uniform particle size distribution.

In some embodiments, the solid composition comprises particles having nonuniform particle size distribution. In some embodiments, the solid composition comprises particles having bimodal particle size distribution.

In some embodiments, the percent mass of the interleukin-1 antagonist in the solid composition is between about 1% and 40%, 40% and 70%, or more than 70%.

In some embodiments, the solid composition comprises a plurality of particles enclosed in a plurality of receptacles. In some embodiments, the receptacles are selected from the group consisting of capsules, blisters, and film covered containers. In some embodiments, the delivery device is suitable for direct administration of the pharmaceutical composition to bronchioles. In some embodiments, the delivery device is suitable for direct administration of the pharmaceutical composition to alveolar tissue.

In yet another aspect, a method of treating an inflammatory disorder of the respiratory tract is disclosed, including administering to a patient in need thereof the pharmaceutical composition according to any one of the embodiments described herein.

### Inflammatory Disorders and Pharmaceutical Composition Administration

In some embodiments, the inflammatory disorder of the respiratory tract is an inflammatory disorder of the upper airways. In some embodiments, the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), bronchiolitis obliterans syndrome (BOS), idiopathic pulmonary fibrosis (IPF), pneumonitis, primary graft dysfunction (PGD), and reperfusion injury.

In some embodiments, the toxic-inhalation lung injury is caused by inhalation of one or more chemical warfare agents. In some embodiments, the chemical warfare agent is selected from the group consisting of chlorine gas and sulfur mustard. In some embodiments, the toxic-inhalation lung injury is chlorine-induced bronchiolitis obliterans syndrome (BOS) and sulfur mustard-induced bronchiolitis obliterans syndrome (BOS).

In some embodiments, the toxic-inhalation lung injury is caused by inhalation of one or more environmental and/or industrial toxic agents. In some embodiments, the environmental and industrial toxic agents are selected from the group consisting of isocyanate, nitrogen oxide, morpholine, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes.

In some embodiments, the toxic-inhalation lung injury is pneumoconiosis or bronchiolitis obliterans. In some embodiments, the toxic-inhalation lung injury is a vaping-associated lung injury. In some embodiments, the vaping-associated lung injury is caused by inhalation of one or more agents selected from the group consisting of diacetyl, α-Tocopheryl acetate, 2,3-pentanedione, nicotine, carbonyls, benzene, toluene, metals, bacterial endotoxins, and fungal glucans.

In some embodiments, the inflammatory disorder is an inflammatory disorder of the lung. In some embodiments, the inflammatory disorder of the respiratory tract is an inflammatory disorder of the lower airways.

In some embodiments, a sustained exposure of the pharmaceutical composition in a lung epithelial lining fluid is between about 15 hours and about 100 hours. In some embodiments, the sustained exposure of the pharmaceutical composition in the lung epithelial lining fluid is at least 24 hours.

In some embodiments, the pharmaceutical composition is administered between about once per week and about three times per day. In some embodiments, the pharmaceutical composition is administered about once or twice daily.

In some embodiments, the pharmaceutical composition is administered via inhalation for between about 3 minutes and about 20 minutes.

In some embodiments, the pharmaceutical composition is administered at a dose of between about 0.5 mg/kg and about 2 mg/kg.

In some embodiments, the pharmaceutical composition binds with substantially similar affinity as an endogenous IL-1β ligand to an IL-1 type 1 receptor.

### EXAMPLES

### Example 1. Inhalation Formulation Screening

To screen the inhalation formulations, the acceptable targeted solution formulation pH for the pulmonary route of administration is between pH 5-8. The solution osmolality is within physiological ranges (˜300 mOsm/kg). Excipients used are “acceptable” or “well characterized” by the pulmonary route and within the concentration ranges/doses listed within the FDA Inactive Ingredient List for approved pulmonary products. Preference is given to either parenteral grade excipients (if available) and/or inhalation grade excipients currently used in marketed products for inhalation in major markets, including the US, EU, and Japan. A tiered approach is used to evaluate the preformulations including a physical stability screening study, stress stability screening study, and a formulation filtration study.

In conducting the preformulation studies, screening studies are conducted to identify formulation matrices and stable ALTA-2530 solutions for nebulization to be used in aerosol characterization studies. ALTA-2530 is a human recombinant IL-1 receptor inhibitor (hIL-1Ra). A control formulation (Kineret) is used as a reference. Formulation components include, but are not be limited to buffers, stabilizers, and tonicity modifiers. The buffers include histidine, phosphate, succinate, glutamate, citrate, PBS, and pyrophosphate. The stabilizers include polysorbate 20 and 80 and other compatible nonionic surfactants, EDTA disodium, glycerin, mannitol, and trehalose. The tonicity modifiers include sodium chloride and dextrose.

In conducting physical stability screening studies, approximately 10 formulations (various matrices+ALTA-2530 plus a Kineret control) using stressed conditions (e.g., freeze/thaw, agitation) are screened to identify potential protein formulation matrices to be used in a preclinical tolerability study. Characterization and output include physical and chemical characterization analyses of approximately 10 formulations (with ALTA-2530) (i.e., appearance, related substances, SEC, DSC, turbidity, DLS) after 1 to 2 freeze/thaw exposure(s) and agitation cycles.

In conducting stress stability screening studies, a solution formulation for use in GLP studies is evaluated using short term temperature/time stress-based stability.

In conducting formulation filtration studies, a lead and a back-up formulation is identified (up to 4 compositions; 2 matrices×2 concentrations) in the stressed testing screening studies. Filter compatibility studies (i.e., impurities and loss of content) using a maximum of 2×0.2 μm filter types is conducted. Results are generated using single and double filtration.

### Example 2. Aerosol Characterization

Using formulations identified in preformulation screening studies from Example 1, stability to nebulization over the in-use period (T=0 and T=24 hr) and dosing duration to simulate clinical dosing using the InnoSpire Go nebulizer is determined. A single nebulizer charge volume for these studies is also determined. Samples are evaluated from preclinical study site engineering runs to assess stability to nebulization over the anticipated dosing duration (i.e., duration of dosing for preclinical studies (e.g., 0, 1, 3 hrs)) using the preclinical nebulizer (Aerogen Solo).

To conduct the stability to nebulization study, a minimum of 2 and maximum of 4 solutions for nebulization (as identified during formulation screening studies) using both the clinical and preclinical nebulizers (if different) are characterized. An impurity profile as generated from the preclinical nebulizer over the intended dosing duration and in-use period (samples will be provided from engineering runs conducted at the preclinical study site) is determined. The impurity profile generated from the clinical nebulizer (Philips InnoSpire Go) over the intended dosing duration and in use period is also determined. Pre-nebulization assessment of solution viscosity, density, turbidity, and surface tension data is collected and analyzed. Data is also collected for both nebulizers for each formulation at T=0 and T=24 hrs (solutions stored at refrigerated conditions) to determine: assay and impurities (pre- and post-nebulization by SEC and RP-HPLC); physical characterization (appearance and turbidity pre- and post-nebulization as collected nebulized solutions and solution remaining in nebulizer); VMD and GSD by Spraytec™; liquid output rate (LOR); and report time to empty, sputter, or clog nebulizer as well as the approximate residual volume in the nebulizer at this timepoint.

The pulmonary dose and dose variability using a minimum of 3 Philips InnoSpire Go nebulizer units over the in-use period for a maximum of 2 formulations (low and high solution concentrations, same matrix) and at 2 nebulizer charge volumes is estimated by generating APSD and GSD from 3 nebulizers; generating DD data (n=10) at a fixed duration (pre-determined time to sputter) using USP 1601; estimating the lung dose (using DD and cut-off of 5 μm and 3.5 μm APSD) and the dose variability; and estimating the lung dose as a function of multiple nebulizer charges for each fixed nebulizer charge volume.

### Example 3. Inhaled Delivery of ALTA-2530 Achieves Extensive and Prolonged Pulmonary Exposure of RhIL-1Ra Compared to Low Level and Transient Exposure Following Bolus IV Injection

ALTA-2530 is a novel inhaled formulation of recombinant human IL-1 receptor antagonist (rhIL-1Ra) in development in some embodiments for bronchiolitis obliterans syndrome (BOS). IL-1 overexpression in BOS drives chronic inflammation and fibroblast activation leading to airway remodeling and impaired oxygen transfer. Endogenous IL-1Ra is upregulated in response to IL-1 to limit cytokine signaling, but expression is inadequate to prevent BOS. Pharmacological IL-1 blockade is considered akin to restoration of physiologic immune regulation.

### Purpose:

To determine if ALTA-2530 is stable during nebulization, achieves aerosol particle diameters consistent with distribution to distal airways, and pulmonary exposure commensurate with treatment of BOS.

### Methods:

Aerosolization and in vivo studies were performed with Aerogen Solo or Philips InnoSpire Go vibrating mesh nebulizers. Rats (n=4/grp/timepoint) received ALTA-2530 by nose-only inhalation (0.63, 1.3, and 2.1 mg/g lung). Serum and bronchioalveolar lavage (BAL) samples were collected for analysis by LC-MSMS. ALTA-2530 in lung epithelial lining fluid (ELF) was calculated using a BALF dilution factor.

Inhaled delivery of ALTA-2530 achieves extensive, stable, and sustained exposure in lung epithelial lining fluid that in rodents markedly exceeds 24 hr, in contrast to exposure following bolus IV delivery where exposure is transient and <20 min. Lung is the target organ for treatment of conditions including, but not limited to: post lung transplant conditions including BOS, primary graft dysfunction (PGD), reperfusion injury, infection related ARDS, or chemical lung injury. Achieving pharmacologically relevant levels of rhIL-1Ra in lung tissue requires high-dose SC or IV treatment with rhIL-1Ra resulting in renal impairment and neutropenia in some patients. IV delivery provide low level and transient exposure to lung tissue. Inhaled delivery targets the organ of clinical significance and achieves long lasting high exposure levels.

Inhaled delivery of ALTA-2530 achieves prolonged pulmonary exposure of rhIL-1Ra that exceeded 24 hr in rat compared to transient exposure of <20 min following bolus IV injection. This is predictive for once or twice daily, or even less frequent, dosing clinically compared to multiple daily IV doses required for the treatment of lung pathologies. Moreover, the ratio of lung epithelial lining fluid to plasma exposures in rats were >2500-fold compared to 0.44-fold for lung tissue: plasma following a 5 hr IV infusion. See Kim et al., Kidney as a major clearance organ for recombinant human interleukin-1 receptor antagonist, Journal of Pharmaceutical Sciences, 1995.

Recombinant human IL-1 receptor antagonist (rhIL-1Ra) exposure was determined in lung bronchioalveolar lavage fluid (BALF) following inhaled delivery to male and female Sprague Dawley rats.

Sprague Dawley male (M) and female (F) rats were weighed and randomized into study groups (Table 1). One group was kept naïve, all other animals were exposed to a single dose of either the Vehicle (normal saline, 0.9% sodium chloride), or to ALTA-2530 test article (TA) recombinant human IL-1 receptor antagonist (rhIL-1Ra) via nose-only inhalation. Target dose levels of rhIL-1Ra were regulated by exposure duration at a target aerosol concentration of 1.5 milligrams (mg)/liter (L).

TABLE 1
Experimental Design

| | | | | Target Necropsy Post Exposure | |
|---|---|---|---|---|---|
| | | Exposure | | 5, 30 minutes, 1, 2, | |
| | Target | Time | | 3, 4, 6, 8, 12 hours | 24, 72 hours |
| Group | Dose | (min) | n | (2M/2F per group) | (3M/3F per group) |
| 1 | Treatment | N/A | 3M/3F | N/A | 24 hour: TK serum, |
| | Naïve | | | | BALF, lung tissue |
| 2 | Vehicle | 180 | 3M/3F | TK serum 1 hr post- | 24 hour: TK serum, |
| | | | | exposure (non-terminal, | BALF, lung tissue |
| | | | | 3M/3F) | |
| 3 | Dose 1 | 60 | 24M/24F | TK serum, BALF, lung | TK serum, BALF, lung |
| | | | | tissue | tissue, Clinical pathology |
| 4 | Dose 2 | 120 | 24M/24F | TK serum, BALF, lung | TK serum, BALF, lung |
| | | | | tissue | tissue, Clinical pathology |
| 5 | Dose 3 | 180 | 24M/24F | TK serum, BALF, lung | TK serum, BALF, lung |
| | | | | tissue | tissue, Clinical pathology |

N/A = not applicable

Blood (serum) and BALF were collected for toxicokinetic (TK) analysis from all TK animals during scheduled necropsy following exposures.

Serum and BALF levels of rhIL-1Ra were determined by means of LC-MSMS. rhIL-1RA was captured from serum and BALF samples using streptavidin magnetic beads coated with anti-human IL-1RA antibody, subjected to “on-bead” proteolysis with trypsin, denatured, reduced, and alkylated, resulting in characteristic peptide fragments originating from rhIL-1RA. A selected characteristic peptide was quantified as a surrogate of the ALTA-2530 concentrations in samples.

Concentrations of rhIL-1Ra in BALF were corrected for the dilution factor introduced during collection of epithelial lining fluid (ELF) using normalization of BALF and plasma urea as described by Rennard et al., J Applied Physiol, 1986. Levels of urea in BALF were less than the lower limit of quantitation (LLOQ) so the normalization factor was calculated using the LLOQ value (1 mg/dL). Thus, the reported values for rhIL-1Ra in ELF are likely an under-estimate of true concentration. Mean plasma urea concentrations were used based on combined gender groups mean values for plasma urea.

### Results:

Nebulized ALTA-2530 delivered rhIL-1Ra particles with mass median aerodynamic diameters of ˜2.5-4 μm, consistent with delivery to small bronchioles. Impurity profiling by HPLC-UV and HPLC-SEC methods and an in vitro potency assay demonstrated rhIL-1Ra protein was stable during nebulization and retained full potency.

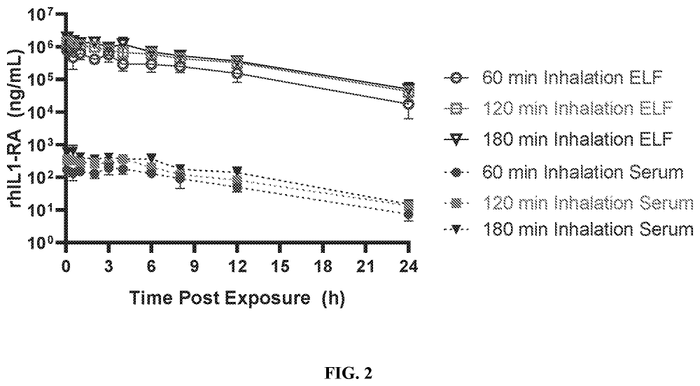

Descriptive pharmacokinetic parameters for rhIL-1Ra in serum and ELF are presented in Table 2. FIG. 2 shows concentration versus time profiles for rhIL-1Ra in ELF and serum following single doses to rat.

TABLE 2
Descriptive pharmacokinetic parameters in serum and
ELF following single doses of ALTA-2530 to rat

| Group #, | Dose^{a} | | Tₘₐₓ | Cₘₐₓ | AUCₗₐₛₜ | AUC_{0-inf} | t_{1/2} |
|---|---|---|---|---|---|---|---|
| Dose level | (mg/kg) | Sample | (h) | (ng/mL) | (h*ng/mL) | (h*ng/mL) | (h) |
| 3, Dose 1 | 3.34 | Serum | 4.0 | 202 | 1785 | — | 4.38 |
| | | ELF | 0.08 | 754,000 | 4,990,000 | 5,430,000 | 6.36 |
| 4, Dose 2 | 7.07 | Serum | 0.08 | 400 | 3,040 | — | 4.77 |
| | | ELF | 0.08 | 1,400,000 | 9,790,000 | 1,400,000 | 5.48 |
| 5, Dose 3 | 11.20 | Serum | 0.50 | 626 | 4,525 | — | 4.14 |
| | | ELF | 0.08 | 1,920,000 | 12,500,000 | 13,700,000 | 4.21 |

^{a}Dose is average pulmonary deposited dose based on terminal body weight with deposition
— Not calculated

### Discussion:

Inhaled delivery of ALTA-2530 achieved prolonged pulmonary exposure of rhIL-1Ra that exceeded 24 hr in rat compared to transient exposure of <20 min following bolus IV injection. Cawthorne assesses PET imaging and γ-counting of lung tissue following a bolus IV dose of [18F]IL-1Ra, which is incorporated by references in its entirety herein. See Cawthorne et al., Biodistribution, pharmacokinetics and metabolism of interleukin-1 receptor antagonist (IL-1RA) using [18F]-ILIRA and PET imaging in rats, B.J. Pharmacology, 2010.

The prolonged exposure of rhIL-1Ra in lung following inhaled delivery of ALTA-2530 is predictive for once or twice daily, or less frequent, dosing clinically compared to multiple daily IV doses required for the treatment of lung pathologies.

The ratio of lung epithelial lining fluid to plasma exposures as AUC were >2500-fold across all inhaled doses compared to 0.44-fold for lung tissue: plasma following a 5 hr IV infusion. See Kim et al., 1995.

IL-1Ra binds to the IL-1RI receptor with comparable affinity as IL-1b; thus, rhIL-1Ra levels ˜100× levels are needed for pharmacological levels in lung tissue. At human equivalent doses (based on mg/g lung) rat BALF rhIL-1ra concentrations exceeded those of IL-1b reported in BAL of BOS patients by >1000×.

Nebulized ALTA-2530 delivers stable and active rhIL-1Ra protein, in a particle size for delivery to small airways of the lung and exposure duration predictive for once daily therapeutic dosing in BOS.

Effective animal doses from in vivo studies (e.g., see Table 2 above) can be converted to appropriate human doses using conversion methods known in the art. See Tepper et al., Breathe in, breath out, it's easy: What you need to know about developing inhaled drugs”, Int J of Tox, 2016 35(4) 376-392. In some embodiments, the rat dose can be converted to human dose based on mg of ALTA-2530 per g of lung weight. In some embodiments, human patients are administered inhaled ALTA-2530 at doses of between about 0.5 mg/kg to about 2 mg/kg.

A nebulized ALTA-2530 delivered to small airways of the lung sustained pharmacologically-relevant levels of rhIL-1Ra protein, demonstrating promise for therapeutic dosing in chronic lung allograft dysfunction. ALTA-2530 rhIL-1Ra was shown to be stable and retained potency after nebulization. ALTA-2530 rhIL-1Ra was shown to be stable in lung ELF. ALTA-2530 formulation achieved extensive and prolonged exposure in ELF that at trough 24 hr after dosing, was >29-fold the rhIL-1Ra IC₅₀ (commercially available IL-1Ra potency assay was used for IC₅₀ determination). The Mass Medium Aerodynamic Diameter (MMAD) from rodent study ranged from 2.18 to 3.19 Thus, inhaled ALTA-2530 delivered IL-1Ra to small airways in nonhuman primates consistent with treatment target for BOS.

### Example 4. Characterizing an rhIL-1Ra for Nebulization

Rodents were administered nebulized ALTA-2530 and results for one nebulization, two nebulization, and three nebulization doses are shown in Tables 3-5, respectively.

TABLE 3
Result (one neb)

| | | Total | API | | | | |
|---|---|---|---|---|---|---|---|
| | | Aerosol | Aerosol | | | | Neb |
| | | Conc. | Conc. | | MMAD | Pre/Post | output |
| Chamber | Filter | (mg/L) | (mg/L) | API | (μm) | Neb Conc. | rate |
| System | Type | (% RSD) | (% RSD) | (%) | (GSD) | (mg/mL) | (mL/min) |
| Rodent | GF/A | 1.09 (16.0) | 0.67 (11.9) | 61 | 3.24 (1.59) | 51.3/53.9 | 0.24 |
| Test #1 | Teflo | 1.14 (19.4) | 0.70 (15.0) | 61 | | | |
| Rodent | GF/A | 1.12 (5.5) | 0.68 (7.1) | 61 | 2.61 (1.64) | 55.7/101.2 | 0.25 |
| Test #2 | Teflo | 1.07 (4.3) | 0.72 (5.3) | 68 | | (?) | |
| NHP | GF/A | 0.89 (23.1) | 0.56 (28.7) | 62 | 3.93 (1.56)¹ | 53.0/54.7 | 0.24 |
| | Teflo | 0.79 (9.6) | 0.50 (11.6) | 64 | 2.42 (1.75)² | | |
| | | | | | 2.61 (1.64)³ | | |

¹the original NHP impactor was likely overloaded repeated samples were collected.
²Repeated sample with In-Tox impactor, reduced sample time
³Repeated sample with NGI

TABLE 4
Result (two nebs)

| | | Total | API | | | | |
|---|---|---|---|---|---|---|---|
| | | Aerosol | Aerosol | | | | |
| | | Conc. | Conc. | | MMAD | Pre/Post Neb Conc. | |
| Chamber | Filter | (mg/L) | (mg/L) | API | (μm) | (mg/mL) | |
| System | Type | (% RSD) | (% RSD) | (%) | (GSD) | Neb #1 | Neb #2 |
| Rodent #1 | GF/A | 1.53 (6.3) | 0.94 (6.4) | 62 | 3.39 (1.51)¹ | 51.1/50.8 | 50.5/66.1 |
| | Teflo | 1.83 (4.6) | 1.15 (3.7) | 63 | 2.34 (1.53)² | | |
| Rodent #2³ | GF/A | 1.57 (8.3) | 0.98 (9.4) | 62 | 2.21 (1.56)² | 52.1/52.3 | 50.0/52.9 |
| | Teflo | 1.97 (1.6) | 1.30 (2.6) | 66 | | | |
| NHP #1 | GF/A | 0.79 (5.5) | 0.50 (5.7) | 61 | 2.83 (1.51)¹ | 49.8/52.3 | 50.3/51.5 |
| (9.5LPM) | Teflo | 0.89 (3.5) | 0.59 (3.8) | 68 | 2.99 (1.51)² | | |
| NHP #2 | GF/A | 1.06 (6.7) | 0.70 (5.7) | 66 | 3.34 (1.54)¹ | 50.6/51.3 | 50.2/49.8 |
| (14LPM) | Teflo | 0.97 (9.1) | 0.65 (8.6) | 67 | 2.75 (1.57)² | | |
| NHP #3 | GF/A | 1.00 (2.4) | 0.66 (0.6) | 66 | 3.10 (1.56)¹ | N/A | N/A |
| (18LPM) | Teflo | 0.95 (3.2) | 0.60 (4.7) | 64 | | | |
| NHP #4 | GF/A | 1.10 (7.5) | 0.71 (7.8) | 65 | N/A | N/A | N/A |
| (18LPM)³ | Teflo | 0.95 (2.6) | 0.64 (4.2) | 68 | | | |
| NHP #5 | GF/A | 1.10 (7.2) | 0.73 (8.1) | 66 | 2.45 (1.54)² | 51.9/52.3 | 52.4/51.8 |
| (18LPM)³ | Teflo | 1.10 (4.1) | 0.74 (3.2) | 67 | | | |

¹In-Tox impactor
²NGI
³Two highest output rate (0.28 & 0.26 mL/min) nebulizers were used. Chamber inlet 90 degree bend was removed

TABLE 5
Result (three nebs)

| | | Total | API | | | Pre/Post Neb Conc. | | |
|---|---|---|---|---|---|---|---|---|
| | | Aerosol | Aerosol | | | (mg/mL) | | |
| | | Conc. | Conc. | | MMAD | Neb #1 | Neb #2 | Neb #3 |
| Chamber | Filter | (mg/L) | (mg/L) | API | (μm) | 0.32 | 0.27 | 0.13 |
| System | Type | (% RSD) | (% RSD) | (%) | (GSD) | mL/min | mL/min | mL/min |
| NHP #1 | GF/A | 1.25 (9.5) | 0.82 (11.6) | 65 | 2.65 (1.55)² | 49.8/51.7 | 50.6/51.4 | 50.8/50.3 |
| (18 LPM)¹ | Teflo | 1.20 (0.7) | 0.80 (0.7) | 67 | | | | |

¹Chamber inlet 90 degree bend was removed
²NGI

As shown in Table 6, characterization of protein by means of an impurity profiling HPLC-UV method indicated the rhIL-1Ra was stable during nebulization. Biological activity, measured as in vitro inhibitory activity, was similar before and after nebulization.

TABLE 6
ALTA-2530 Characterization Data Summary

| | |
|---|---|
| Nebulizer | |
| API Source | ALTA-2530 |
| Inhalation Solution Concentration | 50 mg/mL |
| Nebulizer Charge Volume (mL) | 6 |
| Appearance | Clear colorless solution |
| Physicochemical Properties | |
| pH | 6.3 |
| Osmolality (mOsm/kg) | 303 |
| Surface Tension (mN/m) | 32.8 |
| Density (g/mL) | 1.05 |
| Viscosity (mPa · s) | 1.144 |
| Aerosol Performance | |
| Delivered Dose Results (n = 5) | |
| Actual Solution Concentration (mg/mL) | NR |
| Actual Nebulizer Charge (mg) | 281.90 |
| Delivered Dose (mg) | 252.05 |
| DD (% of charge dose) | 89.4% |
| Estimated Residual Volume | 0.49 |
| in Nebulizer (mL) | |
| APSD by NGI Data (n = 3) | |
| MMAD (μm) | 4.70 |
| GSD | 1.79 |
| FPD < 5.0 μm (mg) | 131.01 |
| FPD < 3.3 μm (mg) | 66.63 |
| Fine Particle Fraction < 5.0 μm (%) | 52.9 |
| Fine Particle Fraction < 3.3 μm (%) | 27.0 |
| Delivered Dose by NGI (mg) | 247.23 |
| Purity by RP-HPLC (% anakinra) | |
| Pre Nebulization | 73.3% |
| Post Nebulization (dose sample) | 73.9% |
| Post Nebulization (retained sample) | 72.5% |
| Change in Solution Concentration Pre- | — |
| Nebulization vs Post-Nebulization | |
| Residual Solution Concentration (% change) | |
| Purity by SEC (% anakinra monomer) | |
| Pre-Nebulization | 99.30 |
| Post Nebulization (dose sample) | 99.30 |
| Post Nebulization (retained sample) | 99.60 |
| Bioactivity IC₅₀ Values (μg/mL) | Paras rhIL-1Ra, as |
| | received IC₅₀ = 0.3765 |
| Pre-Nebulized (Control) | 0.23 |
| Nebulized Dose | 0.4439 |
| | 0.5458 |
| | 0.6 |
| Residual Dose in Nebulizer | 0.489 |
| | 0.6057 |
| Post-Nebulized (Control) | — |

### Example 5. Physical and Aerosol Characterization of Alternate Sources of Anakinra

Physical and aerosol characterization studies were performed on alternate sources of anakinra. Physical and aerosol characterization were performed on two sources of Anakinra from Sobi (Kineret®) and Paras. Aerosol test methods were used to characterize inhalation solutions containing anakinra at 50 mg/mL when nebulized with a Philips InnoSpire Go vibrating mesh nebulizer.

Based on the results from this study, the 50 mg/mL inhalation solutions when nebulized using the InnoSpire Go were shown to be stable to nebulization by SEC, RP-HPLC and activity assays. A summary of the key results is shown in Table 7. It should be noted that there were significant differences observed in the starting purity of the Sobi and Paras Anakinras. The Sobi Anakinra showed higher purity as analyzed by RP-HPLC, whereas the Paras anakinra showed higher purity of Anakinra monomer as analyzed by SEC. The MMAD values from both protein solutions were similar, but did not meet the target acceptance criteria (˜4.7 μm compared with target of NMT 4 μm). However it is noted that at 4.7 μm, the MMAD value observed is within the 2-5 μm range considered optimal for local pulmonary delivery to the bronchioles.

TABLE 7
Summary of Characterization of Alternative Sources of Anakinra

| | API Source | |
|---|---|---|
| Attribute | Sobi | Paras |
| Appearance | Clear | Clear |
| | colorless | colorless |
| | solution | solution |
| pH | 6.3 | 6.3 |
| Osmolality (mOsm/kg) | 296 | 303 |
| Surface Tension (mN/m) | 37.8 | 32.8 |
| Viscosity (mPa · s) | 1.061 | 1.144 |
| Delivered Dose (mg) | 248.55 | 252.05 |
| MMAD (μm) | 4.75 | 4.70 |
| GSD | 1.75 | 1.79 |
| FPD < 5.0 μm (mg) | 121.86 | 131.01 |
| FPD < 3.3 μm (mg) | 59.82 | 66.63 |
| Fine Particle Fraction < 5.0 μm (%) | 52.2 | 52.9 |
| Fine Particle Fraction < 3.3 μm (%) | 25.6 | 27.0 |
| Purity by RP-HPLC (% anakinra) | | |
| Pre Nebulization: | 90.0 | 73.3 |
| Post Nebulization (Dose Sample): | 89.9 | 73.9 |
| Post Nebulization (Retained Sample): | 88.1 | 72.5 |
| Purity by SEC (% anakinra monomer) | | |
| Pre-Nebulization: | 95.6 | 99.3 |
| Post Nebulization (Dose Sample): | 95.7 | 99.3 |
| Post Nebulization (Retained Sample): | 96.1 | 99.6 |
| Bioactivity (IC₅₀ μg/mL) | Kineret | Paras |
| | Anakinra, | Anakinra, |
| | as received | as received |
| | IC₅₀ = 0.5966 | IC₅₀ = 0.3765 |
| Initial | IC₅₀ = 0.3865 | IC₅₀ = 0.2300 |
| Protein Post DD Run 1 | IC₅₀ = 0.5406 | IC₅₀ = 0.4439 |
| Protein Post DD Run 2 | IC₅₀ = 0.5675 | IC₅₀ = 0.6000 |
| Protein Post Neb Run 1 | IC₅₀ = 0.5947 | IC₅₀ = 0.4890 |
| Protein Post Neb Run 2 | IC₅₀ = 0.8023 | IC₅₀ = 0.6057 |
| Protein Post DD run 1 (Duplicate) | IC₅₀ = 0.5688 | IC₅₀ = 0.5458 |

The aerodynamic particle size distribution was determined on the Anakira inhalation solutions when nebulized with a Philips InnoSpire Go nebulizer per TM-286-001-05. The method involves collecting the mist from the nebulization into a Next Generation Impactor (NGI), which consists of 7 stages plus a filter. A flow rate of 15 LPM was used, and the NGI was placed into a 2-8° C. refrigerator for at least 90 minutes prior to testing. A fill volume of 6.0 mL of inhalation solution was used. NGI testing was performed for n=3 replicates of both Anakinra inhalation solutions. The NGI components were extracted according to the test method. After the nebulization was complete the residual volume in the nebulizer was ˜0.5 mL, where droplets were distributed across the nebulizer. The actual volume remaining in the reservoir was <0.5 mL and was not a sufficient volume that could be removed. The nebulizer was placed in a plastic bag along with the diluent for the extraction procedure. The results for the n=3 replicates of the Paras and Sobi inhalation solutions are shown in Tables 8 and 9, respectively.

TABLE 8
Particle Size by NGL 50 mg/mL Paras API Solution

| | Deposition (mg) | | | | | |
|---|---|---|---|---|---|---|
| | Replicate 1 | Replicate 2 | Replicate 3 | | | |
| Component | Neb ID = C | Neb ID = C | Neb ID = C | Mean | SD | % RSD |
| Induction Port | 3.00 | 2.16 | 2.22 | 2.46 | 0.47 | 19 |
| Stage 1 | 11.86 | 9.57 | 10.04 | 10.49 | 1.21 | 12 |
| Stage 2 | 26.03 | 23.01 | 24.73 | 24.59 | 1.51 | 6 |
| Stage 3 | 69.38 | 63.75 | 58.62 | 63.92 | 5.38 | 8 |
| Stage 4 | 95.73 | 71.09 | 70.62 | 79.15 | 14.36 | 18 |
| Stage 5 | 54.40 | 40.82 | 44.68 | 46.63 | 7.00 | 15 |
| Stage 6 | 17.62 | 14.62 | 12.82 | 15.02 | 2.43 | 16 |
| Stage 7 | 2.35 | 4.53 | 5.49 | 4.12 | 1.61 | 39 |
| Filter | 1.28 | 1.03 | 0.26 | 0.86 | 0.53 | 62 |
| Nebulizer | 24.89 | 25.14 | 23.50 | 24.51 | 0.89 | 4 |
| (Residual Drug) | | | | | | |
| Parameter | Replicate 1 | Replicate 2 | Replicate 3 | Mean | SD | % RSD |
| MMAD (μm) | 4.60 | 4.79 | 4.71 | 4.70 | 0.10 | 2 |
| GSD | 1.72 | 1.82 | 1.83 | 1.79 | 0.06 | 3 |
| FPD <5.0 μm (mg) | 153.51 | 118.82 | 120.69 | 131.01 | 19.51 | 15 |
| FPD <3.3 μm (mg) | 75.65 | 60.99 | 63.25 | 66.63 | 7.89 | 12 |
| Fine Particle | 54.5 | 51.5 | 52.6 | 52.9 | 1.51 | 3 |
| Fraction <5.0 μm (%) | | | | | | |
| Fine Particle | 26.9 | 26.5 | 27.6 | 27.0 | 0.56 | 2 |
| Fraction <3.3 μm (%) | | | | | | |
| Delivered Dose (mg) | 281.65 | 230.57 | 229.48 | 247.23 | 29.81 | 12 |
| Amount Retained | 24.89 | 25.14 | 23.50 | 24.51 | 0.89 | 4 |
| in Nebulizer after | | | | | | |
| Dosing (Residual Drug) | | | | | | |
| Recovery (%) | 109.6 | 112.8 | 112.9 | 111.8 | 1.86 | 2 |

TABLE 9
Particle Size by NGL 50mg/mL Sobi API Solution

| | Deposition (mg) | | | | | |
|---|---|---|---|---|---|---|
| | Replicate 1 | Replicate 2 | Replicate 3 | | | |
| Component | Neb ID = B | Neb ID = C | Neb ID = C | Mean | SD | % RSD |
| Induction Port | 2.73 | 2.38 | 2.52 | 2.54 | 0.17 | 7 |
| Stage 1 | 11.25 | 9.88 | 10.15 | 10.42 | 0.72 | 7 |
| Stage 2 | 30.08 | 24.28 | 26.28 | 26.88 | 2.95 | 11 |
| Stage 3 | 56.14 | 57.77 | 56.85 | 56.92 | 0.82 | 1 |
| Stage 4 | 69.63 | 77.29 | 81.91 | 76.28 | 6.20 | 8 |
| Stage 5 | 34.44 | 45.11 | 43.30 | 40.95 | 5.71 | 14 |
| Stage 6 | 10.53 | 15.09 | 14.47 | 13.36 | 2.48 | 19 |
| Stage 7 | 3.94 | 5.33 | 5.02 | 4.76 | 0.73 | 15 |
| Filter | 0.81 | 0.24 | 1.19 | 0.75 | 0.48 | 64 |
| Nebulizer | 38.45 | 35.05 | 35.60 | 36.36 | 1.82 | 5 |
| (Residual Drug) | | | | | | |
| Parameter | Replicate 1 | Replicate 2 | Replicate 3 | Mean | SD | % RSD |
| MMAD (μm) | 5.02 | 4.60 | 4.63 | 4.75 | 0.23 | 5 |
| GSD | 1.76 | 1.77 | 1.73 | 1.75 | 0.02 | 1 |
| FPD <5.0 μm (mg) | 106.35 | 128.64 | 130.60 | 121.86 | 13.47 | 11 |
| FPD <3.3 μm (mg) | 49.71 | 65.78 | 63.98 | 59.82 | 8.80 | 15 |
| Fine Particle | 48.4 | 54.2 | 54.0 | 52.2 | 3.28 | 6 |
| Fraction <5.0 μm (%) | | | | | | |
| Fine Particle | 22.6 | 27.7 | 26.5 | 25.6 | 2.64 | 10 |
| Fraction <3.3 μm (%) | | | | | | |
| Delivered Dose (mg) | 219.53 | 237.38 | 241.68 | 232.87 | 11.75 | 5 |
| Amount Retained | 38.45 | 35.05 | 35.60 | 36.36 | 1.82 | 5 |
| in Nebulizer after | | | | | | |
| Dosing (Residual Drug) | | | | | | |
| Recovery (%) | 98.6 | 99.0 | 100.7 | 99.4 | 1.09 | 1 |

The aerosol performance of the alternate sources of anakinra against the target acceptance criteria is show in Table 10.

TABLE 10
Aerosol Performance of Alternate Sources of Anakinra

| | | | Target Acceptance |
|---|---|---|---|
| Parameter | Sobi API | Paras API | Criteria |
| Nebulization time | 10 to 14 | 9 to 14 | Between 5 and 30 |
| (min) | | | minutes |
| Drug retained in | 30.59 | 24.32 | Report results |
| nebulizer for Dose | | | |
| Testing (mg) | | | |
| Delivered Dose (mg) | 248.55 | 252.05 | Report mean ED |
| | No values | No values | Individuals |
| | outside ± 35 | outside ± 35 | within +/− 35% |
| | of the mean | of the mean | of the mean ED |
| Delivered Dose % | 101.5% | 98.0% | 85-115% |
| Mass Balance | | | |
| Drug retained in | 36.36 | 24.51 | Report results |
| nebulizer for NGI | | | |
| Testing (mg) | | | |
| NGI % Mass Balance | 99.4% | 111.8% | 85-115% |
| MMAD (μm) | 4.75 | 4.70 | NMT 4 um |
| GSD | 1.75 | 1.79 | NMT 3 |
| FPD < 5.0 μm (mg) | 121.86 | 131.01 | Report Results |
| FPD < 3.3 μm (mg) | 59.82 | 66.63 | Report Results |
| | | | Target: 10 mg |

Experiments were conducted to determine the in vitro anakinra activity of test samples using the DiscoverX, PathHunter® Anakinra Bioassay Kit (Cat #93-1032Y3-00105). The protocol in DiscoverX, PathHunter® Anakinra Bioassay Manual was followed. The kit provided bioassay cells will be thawed using 1 mL of pre-warmed CP5, resuspended in a total of 19.2 mL of CP5, and plated in 80 μL in a 96-well plate per manufacture's protocol and incubated overnight (24 hours) in a humidified incubator at 37° C. with 5% CO2 and 95% air. Samples were added in combination with the kit provided inhibitor and incubated for 6 hours in a humidified incubator at 37° C. with 5% CO2 and 95% air. 10 μL Detection Reagent 1 was added to each well and incubated for 15 minutes at room temperature in the dark. 40 μL of Detection Reagent 2 was added to each well and incubated for 60 minutes at room temperature in the dark. Plates were then read for Chemiluminescence on the Biotech Synergy II microplate reader. Tests will be run as two separate experiments for Kineret-base plate and Paras-based plate. Results are shown below in Tables 11 and 12.

TABLE 11
Protein K Test Agents Titration
([Inhibitor vs. response - Variable slope (four parameters)

| | Bottom | Top | Hill | IC₅₀ |
|---|---|---|---|---|
| | Asymptote | Asymptote | Slope | (μg/mL) |
| Kineret | 8456 | 43606 | 1.238 | 05966 |
| Protein K Initial | 7898 | 48308 | 09043 | 0 3865 |
| Protein K Post | 8354 | 46373 | 1.149 | 0 5406 |
| DD run 1 | | | | |
| Protein K Post | 9449 | 42834 | 1.341 | 0.5675 |
| DDrun2 | | | | |
| Protein K Neb run 1 | 9540 | 43684 | 1.221 | 0.5947 |
| Protein K Neb run 2 | 9474 | 41796 | 1.552 | 0.8023 |
| Protein K Post | 8633 | 45724 | 1.15 | 0.5688 |
| DD run 1-duplicate | | | | |

TABLE 12
Protein P Test Agents Titration
([Inhibitor] vs. response - Variable slope (four parameters)

| | Bottom | Top | Hill | IC₅₀ |
|---|---|---|---|---|
| | Asymptote | Asymptote | Slope | (μg/mL) |
| Paras | 9236 | 43906 | 1.481 | 0.3765 |
| Protein P Initial | S321 | 44828 | 1.742 | 023 |
| Protein P Post | 8058 | 42449 | 1.267 | 04439 |
| DD run 1 | | | | |
| Protein P Post | 767S | 40191 | 1.136 | 060 |
| DD run 2 | | | | |
| Protein P Neb run 1 | 9642 | 43298 | 1 576 | 0489 |
| Protein P Neb run 2 | 9307 | 42869 | 1.545 | 06057 |
| Protein P Post | 9989 | 42160 | 1.848 | 0.5458 |
| DD ran 1-duplicate | | | | |

### Example 6. Development and Characterization of Anakinra Nebulized Solution

Nebulized solutions of anakinra were developed and characterized. A screening study was designed and executed to characterize and evaluate the stability of nebulized solutions prepared from Kineret®, an IV formulation of anakinra. Based on the results from this study, Kineret diluted to 5 and 50 mg/mL and nebulized using the AeroEclipse II Jet Nebulizer were shown to be stable to nebulization by SEC, RP-HPLC and bioactivity assays. The concentration of the anakinra solution remaining in the nebulizer was higher post nebulization than the pre nebulization solution concentration, which is due to the recirculation process associated with the jet nebulizer during dosing. The MMAD of the nebulized solutions were greater than the target acceptance criteria; however it is noted that the MMAD values observed are within the 2-5 μm range considered optimal for local pulmonary delivery to the bronchioles. A summary of the key results is shown in Table 13.

TABLE 13
Summary of Characterization of Nebulized Anakinra

| Inhalation Solution | 5 mg/mL | 50 mg/mL |
|---|---|---|
| Appearance | Clear colorless | Clear colorless |
| | solution | solution |
| pH | 6.36 | 6.30 |
| Osmolality (mOsm/kg) | 287 | 296 |
| Surface Tension (mN/m) | 34.6 | 37.8 |
| Viscosity (mPa · s) | 0.85 | 1.06 |
| Delivered Dose (mg) | 16.06 | 238.05 |
| MMAD (μm) | 4.97 | 4.92 |
| GSD | 2.14 | 2.07 |
| FPD < 5.0 μm (mg) | 9.58 | 110.33 |
| FPD < 3.3 μm (mg) | 5.70 | 64.36 |
| Fine Particle Fraction < 5.0 μm (%) | 48.8 | 49.5 |
| Fine Particle Fraction < 3.3 μm (%) | 29.0 | 28.8 |
| Purity by RP-HPLC (% anakinra) | 90.1% | 88.9% |
| Pre Nebulization | | |
| Purity by RP-HPLC (% anakinra) | 88.0% | 89.4% |
| Post Nebulization | | |
| Change in Solution Concentration Pre- | +33.2% | +48.0% |
| Nebulizationvs Post-Nebulization | | |
| Residual Solution Concentration | | |
| (% change) | | |
| Purity by SEC (% anakinra monomer) | 94.8% | 96.1% |
| Pre-Nebulization | | |
| Purity by SEC (% anakinra monomer) | 96.4% | 96.1% |
| PostNebulization | | |
| Bioactivity IC₅₀ Values (μg/mL) | 0.5941 | 0.4546 |
| Pre-Nebulized | | |
| Control Nebulized Dose | 1.095 | 0.5938 |
| Residual Dose | 0.6457 | 0.4539 |
| Post-Nebulized Control | 0.5759 | 0.5454 |

Bioactivity results (e.g., see Table 13) are interpreted as follows. The DiscoverX Pathhunter Anakinra bioassay measures the dimerization induced activation of IL1R1/IL1RAP by IL-1B. Anakinra (also known as Kineret) is a biologic agent used to treat rheumatoid arthritis. Anakinra functions by blocking the IL-1B induced dimerization of IL1R1/IL1RAP. In this study, two experiments were conducted. In the first experiment, the dose response of IL-1B for activation of the DiscoverX Pathhunter Anakinra assay was evaluated, and the dose response of Anakinra for inhibition of the assay was also evaluated. IL-1B activated the assay with an EC₅₀ of 0.16 ng/ml. A concentration of 3 ng/ml of IL-1B was chosen to use for the inhibition by Kineret® (100 mg/mL Anakinra for injection). At this concentration of IL-1B, Kineret® Anakinra inhibited the assay with an IC₅₀ of 0.65 μg/ml. In the second experiment, IL-1B at 3 ng/ml was used to activate the assay. The evaluation of the inhibition by Kineret® Anakinra was compared to eight test samples. The repeat IC₅₀ for Kineret® Anakinra, 0.55 μg/ml, was very close to that determined in test 1, 0.65 μg/ml indicating excellent reproducibility of the assay. The pre-nebulized control (sample 1) had an IC₅₀ value of 0.45 μg/ml. The effect of treatment on the Anakinra activity of each sample is reflected in the IC₅₀ obtained. Increases in the IC₅₀ relative to the control represent a loss in Anakinra activity. The test samples had IC₅₀ values ranging from 0.45 μg/ml for sample 3 to 1.10 μg/ml for sample 6. The indicated treatments for samples 2, 3, 4, 5, 7, and 8 had negligible effects on the Anakinra activity of the sample (<20% change relative to sample 1). The treatment of sample 6 reduced the Anakinra activity by 50% relative to sample 5 which is the control for this sample. The experimental results provided are based on a single measurement per treated sample

### Delivered Dose

A method was developed to determine the delivered dose profile of the Anakira inhalation solutions when nebulized with a AeroEclipse II Jet nebulizer per TM-286-001-03. The method involves collecting the mist from the nebulization into a PARI filter pad. A flow rate of 15 LPM and a fill volume of 6.0 mL of inhalation solution were used. Delivered dose samples prepared during the course of the method development were analyzed via HPLC as per the current version of TM-286-001-01 (size exclusion chromatography method) and TM 001-02 (reversed-phase HPLC method). The results for the 8 runs, with the 5 mg/mL inhalation solution, and the 2 runs, with the 50 mg/mL inhalation solutions, are shown in Tables 14 and 15, respectively.

TABLE 14
Delivered Dose Method Development Results, 5 mg/mL Solutions for Nebulization

| | | | | | | Amount | Amount of | | | |
|---|---|---|---|---|---|---|---|---|---|---|
| | | | | | Nebulizer | Inhalation | Anakinra | | | |
| | Run | Nebulizer Weights (g) | | | Fill | Solution | Drug in | Delivered | Residual | |
| | Time | W₁ | W₂ | W₃ | Weight | Delivered | Nebulizer | Dose | Drug | Recovery |
| Replicate | (min) | (Empty) | (Filled) | (Final) | (g) | (g) | (mg) | (mg) | (mg) | (%) |
| 1 | 12 | 30.8334 | 36.8326 | 32.2508 | 5.9992 | 4.5818 | 24.34 | 16.32 | 9.29 | 105.2 |
| 2 | 11 | 30.8613 | 36.8933 | 32.8390 | 6.0320 | 4.0543 | 24.47 | 14.14 | 10.08 | 99.0 |
| 3 | 13 | 30.7505 | 36.8158 | 32.7423 | 6.0653 | 4.0735 | 24.61 | 14.10 | 10.86 | 101.4 |
| 4 | 14 | 30.7089 | 36.7116 | 32.8169 | 6.0027 | 3.8947 | 24.35 | 11.31 | 11.24 | 92.6 |
| 5 | 10 | 30.6958 | 36.7432 | 33.2843 | 6.0474 | 3.4589 | 24.54 | 12.34 | 13.21 | 104.1 |
| 6 | 12 | 30.8539 | 36.8877 | 32.5518 | 6.0338 | 4.3359 | 24.48 | 15.98 | 9.07 | 102.3 |
| 7 | 12 | 30.8359 | 36.7376 | 32.5217 | 5.9017 | 4.2159 | 23.94 | 15.96 | 8.25 | 101.1 |
| 8 | 12 | 30.6976 | 36.6709 | 33.0108 | 5.9733 | 3.6601 | 24.23 | 13.40 | 11.26 | 101.7 |

| | | | |
|---|---|---|---|
| Average | 14.19 | 10.41 | 100.9 |
| Standard Deviation | 1.822 | 1.571 | 3.857 |
| % RSD | 13 | 15 | 4 |

TABLE 15
Delivered Dose Method Development Results, 50 mg/mL Solutions for Nebulization

| | | | | | | Amount | Amount of | | | |
|---|---|---|---|---|---|---|---|---|---|---|
| | | | | | Nebulizer | Inhalation | Anakinra | | | |
| | Run | Nebulizer Weights (g) | | | Fill | Solution | Drug in | Delivered | Residual | |
| | Time | W₁ | W₂ | W₃ | Weight | Delivered | Nebulizer | Dose | Drug | Recovery |
| Replicate | (min) | (Empty) | (Filled) | (Final) | (g) | (g) | (mg) | (mg) | (mg) | (%) |
| 1 | 12 | 30.7516 | 36.7673 | 32.9773 | 6.0157 | 3.7900 | 287.03 | 155.37 | 118.42 | 95.4 |
| 2 | 12 | 30.7104 | 36.7824 | 32.8788 | 6.0720 | 3.9036 | 289.72 | 159.97 | 119.88 | 96.6 |

| | | | |
|---|---|---|---|
| Average | 157.67 | 119.15 | 96.0 |

The results for the 5 runs with the 5 mg/mL inhalation solution and the 5 runs with the 50 mg/mL inhalation solutions are shown in Tables 16 and 17, respectively. The impurities levels present in the nebulizer samples were equivalent (within the error of the HPLC-RP method) to the impurities levels present in the primary filter samples in most cases (replicate 1 was an exception). The impurities levels in the secondary filter were approximately 10-15% higher, however it should be noted that the concentration of anakinra in secondary filter samples was approximately one tenth of the target concentration for impurities in the HPLC-RP method. The low concentration of the sample could adversely impact the quantitation of impurities in the sample.

TABLE 16
Delivered Dose Results, 5 mg/mL Solutions for Nebulization

| | | | | | | Amount | Amount of | | | |
|---|---|---|---|---|---|---|---|---|---|---|
| | | | | | Nebulizer | Inhalation | Anakira | | | |
| | Run | Nebulizer Weights (g) | | | Fill | Solution | Drug in | Delivered | Residual | |
| | Time | W₁ | W₂ | W₃ | Weight | Delivered | Nebulizer | Dose | Drug | Recovery |
| Replicate | (min) | (Empty) | (Filled) | (Final) | (g) | (g) | (mg) | (mg) | (mg) | (%) |
| 1 | 12 | 30.7510 | 36.7750 | 32.6108 | 6.0240 | 4.1642 | 30.28 | 16.37 | 11.25 | 91.2 |
| 2 | 12 | 30.7486 | 36.7662 | 32.6466 | 6.0176 | 4.1196 | 30.25 | 16.69 | 12.09 | 95.1 |
| 3 | 12 | 30.8426 | 36.7733 | 32.9654 | 5.9307 | 3.8079 | 29.82 | 15.04 | 12.29 | 91.7 |
| 4 | 12 | 30.7075 | 36.6578 | 32.7352 | 5.9503 | 3.9226 | 29.91 | 14.28 | 12.40 | 89.2 |
| 5 | 12 | 30.8322 | 36.7467 | 32.3967 | 5.9145 | 4.3500 | 29.73 | 17.90 | 9.73 | 92.9 |

| | | | |
|---|---|---|---|
| Average | 16.06 | 11.55 | 92.0 |
| Standard Deviation | 1.421 | 1.115 | 2.188 |
| % RSD | 9 | 10 | 2 |

| | |
|---|---|
| ±35% of Mean DD | +/−5.62 mg |
| Acceptable DD Range | 10.44 mg-21.68 mg |
| # of Values Outside | 0 |
| of ±35% the Mean | |

TABLE 17
Delivered Dose Results, 50 mg/mL Solutions for Nebulization

| | | | | | | Amount | | | | |
|---|---|---|---|---|---|---|---|---|---|---|
| | | | | | | Inhalation | Amount of | | | |
| | | | | | Nebulizer | Solution | Anakinra | | | |
| | Run | Nebulizer Weights (g) | | | Fill | Delivered | Drug in | Delivered | Residual | |
| | Time | W₁ | W₂ | W₃ | Weight | V | Nebulizer | Dose | Drug | Recovery |
| Replicate | (min) | (Empty) | (Filled) | (Final) | (g) | (g) | (mg) | (mg) | (mg) | (%) |
| 1 | 12 | 30.8334 | 36.9395 | 31.7732 | 6.1061 | 5.1663 | 340.56 | 234.92 | 81.76 | 93.0 |
| 2 | 12 | 30.8418 | 36.9530 | 31.9982 | 6.1112 | 4.9548 | 340.85 | 236.46 | 91.40 | 96.2 |
| 3 | 12 | 30.7075 | 36.7817 | 31.8682 | 6.0742 | 4.9135 | 338.78 | 241.38 | 83.44 | 95.9 |
| 4 | 12 | 30.8623 | 36.9687 | 31.9016 | 6.1064 | 5.0671 | 340.58 | 234.17 | 81.96 | 92.8 |
| 5 | 12 | 30.7500 | 36.8171 | 31.5682 | 6.0671 | 5.2489 | 338.39 | 243.33 | 72.52 | 93.3 |

| | | | |
|---|---|---|---|
| Average | 238.05 | 82.22 | 94.2 |
| Standard Deviation | 4.07 | 6.71 | 1.65 |
| % RSD | 2 | 8 | 2 |

| | |
|---|---|
| +/−35% of Mean DD | +/−83.32 mg |
| Acceptable DD Range | 154.73 mg-321.37 mg |
| # of Values Outside | 0 |
| of ±35% the Mean | |

### Aerodynamic Particle Size Distribution by NGI

A method was developed to determine the aerodynamic particle size distribution of Anakinra inhalation solution when nebulized with an AeroEclipse II Jet nebulizer per TM-286-001-04. The method involves collecting the mist from the nebulization into a Next Generation Impactor (NGI), which consists of 7 stages plus a filter. A flow rate of 15 LPM was used and the NGI was placed into a 2-8° C. refrigerator for at least 90 minutes prior to testing. A fill volume of 6.0 mL of inhalation solution was used. Aerosol samples prepared during the course of the method developed were analyzed via HPLC as per the current version of TM-286-001-01 (size exclusion chromatography method) and TM-286-001-02 (reversed-phase HPLC method). A total of 6 NGI runs were performed using the 5 mg/mL inhalation solution. The results are provided in Table 18. The MMAD ranged from 4.66 to 5.06 μm.

TABLE 18
Particle Size by NGI Method Development 5 mg/mL Solutions for Nebulization

| | Deposition (mg) | | | | | | | | |
|---|---|---|---|---|---|---|---|---|---|
| Component | Replicate 1 | Replicate 2 | Replicate 3 | Replicate 4 | Replicate 5 | Replicate 6 | Mean | SD | % RSD |
| Induction Port | 0.22 | 0.26 | 0.20 | 0.22 | 0.31 | 0.24 | 0.24 | 0.04 | 17 |
| Stage 1 | 0.98 | 0.94 | 0.98 | 1.07 | 1.21 | 1.14 | 1.05 | 0.11 | 10 |
| Stage 2 | 2.23 | 2.11 | 2.11 | 2.51 | 2.54 | 2.54 | 2.34 | 0.21 | 9 |
| Stage 3 | 3.90 | 3.70 | 3.78 | 4.07 | 4.00 | 4.37 | 3.97 | 0.24 | 6 |
| Stage 4 | 4.00 | 4.26 | 3.90 | 4.42 | 3.99 | 4.51 | 4.18 | 0.25 | 6 |
| Stage 5 | 3.01 | 3.10 | 2.75 | 3.11 | 2.78 | 3.07 | 2.97 | 0.16 | 5 |
| Stage 6 | 1.19 | 1.27 | 1.38 | 1.34 | 1.40 | 1.33 | 1.32 | 0.08 | 6 |
| Stage 7 | 0.49 | 0.61 | 0.58 | 0.68 | 0.55 | 0.68 | 0.60 | 0.07 | 13 |
| Filter | 0.07 | 0.09 | 0.11 | 0.11 | 0.10 | 0.13 | 0.10 | 0.02 | 19 |
| Nebulizer | 7.88 | 7.56 | 8.06 | 5.32 | 6.37 | 4.97 | 6.70 | 1.34 | 20 |
| (Residual Drug) | | | | | | | | | |
| Parameter | Replicate 1 | Replicate 2 | Replicate 3 | Replicate 4 | Replicate 5 | Replicate 6 | Mean | SD | % RSD |
| MMAD (μm) | 4.89 | 4.66 | 4.81 | 4.84 | 5.06 | 4.94 | 4.87 | 0.14 | 3 |
| GSD | 2.11 | 2.05 | 2.13 | 2.10 | 2.18 | 2.10 | 2.11 | 0.04 | 2 |
| FPD <5.0 μm (mg) | 8.02 | 8.55 | 8.00 | 8.83 | 8.07 | 8.89 | 8.39 | 0.41 | 5 |
| FPD <3.3 μm (mg) | 4.77 | 5.08 | 4.83 | 5.24 | 4.83 | 5.22 | 4.99 | 0.21 | 4 |
| Fine Particle | 49.8 | 52.3 | 50.6 | 50.4 | 47.8 | 49.3 | 50.0 | 1.48 | 3 |
| Fraction <5.0 μm (%) | | | | | | | | | |
| Fine Particle | 29.6 | 31.1 | 30.5 | 29.9 | 28.6 | 28.9 | 29.8 | 0.94 | 3 |
| Fraction <3.3 μm (%) | | | | | | | | | |
| Delivered Dose (mg) | 16.10 | 16.35 | 15.81 | 17.52 | 16.88 | 18.03 | 16.78 | 0.86 | 5 |
| Amount Retained | 7.88 | 7.56 | 8.06 | 5.32 | 6.37 | 4.97 | 6.70 | 1.34 | 20 |
| in Nebulizer after | | | | | | | | | |
| Dosing (Residual Drug) | | | | | | | | | |
| Recovery (%) | 98.5 | 97.5 | 98.7 | 95.2 | 95.9 | 95.1 | 96.8 | 1.62 | 2 |

The results for the 3 runs with the 5 mg/mL inhalation solution and 3 runs with the 50 mg/mL inhalation solutions are shown in Tables 19 and 20, respectively. The MMAD ranged from 4.80 to 5.09 μm for the 5 mg/mL inhalation solution, while it ranged from 4.62 to 5.50 μm for the 50 mg/mL inhalation solution.

TABLE 19
Particle Size by NGL 5 mg/mL Solutions for Nebulization

| | Deposition (mg) | | | | | |
|---|---|---|---|---|---|---|
| Component | Replicate 1 | Replicate 2 | Replicate 3 | Mean | SD | % RSD |
| Stage 1 | 1.31 | 1.33 | 1.48 | 1.37 | 0.09 | 7 |
| Stage 2 | 2.74 | 2.78 | 3.03 | 2.85 | 0.16 | 6 |
| Stage 3 | 4.51 | 4.63 | 4.66 | 4.60 | 0.08 | 2 |
| Stage 4 | 4.91 | 4.81 | 4.60 | 4.8 | 0.16 | 3 |
| Stage 5 | 3.49 | 3.06 | 3.32 | 3.3 | 0.22 | 7 |
| Stage 6 | 1.54 | 1.53 | 1.47 | 1.5 | 0.04 | 3 |
| Stage 7 | 0.84 | 0.61 | 0.72 | 0.7 | 0.11 | 16 |
| Filter | 0.17 | 0.18 | 0.18 | 0.2 | 0.01 | 4 |
| Nebulizer | 7.67 | 8.27 | 8.05 | 8.0 | 0.31 | 4 |
| (Residual Drug) | | | | | | |
| Parameter | Replicate 1 | Replicate 2 | Replicate 3 | Mean | SD | % RSD |
| MMAD (μm) | 4.80 | 5.02 | 5.09 | 4.97 | 0.15 | 3 |
| GSD | 2.12 | 2.09 | 2.21 | 2.14 | 0.06 | 3 |
| FPD <5.0 μm (mg) | 10.02 | 9.29 | 9.43 | 9.58 | 0.39 | 4 |
| FPD <3.3 μm (mg) | 6.03 | 5.38 | 5.69 | 5.70 | 0.33 | 6 |
| Fine Particle | 50.6 | 48.2 | 47.6 | 48.8 | 1.62 | 3 |
| Fraction <5.0 μm (%) | | | | | | |
| Fine Particle | 30.5 | 27.9 | 28.7 | 29.0 | 1.31 | 5 |
| Fraction <3.3 μm (%) | | | | | | |
| Delivered Dose (mg) | 19.79 | 19.28 | 19.83 | 19.63 | 0.31 | 2 |
| Amount Retained | 7.67 | 8.27 | 8.05 | 7.99 | 0.31 | 4 |
| in Nebulizer after | | | | | | |
| Dosing (Residual Drug) | | | | | | |
| Recovery (%) | 92.4 | 91.6 | 91.9 | 92.0 | 0.44 | 0 |

TABLE 20
Particle Size by NGL 50 mg/mL Solutions for Nebulization

| | Deposition (mg) | | | | | |
|---|---|---|---|---|---|---|
| Component | Replicate 1 | Replicate 2 | Replicate 3 | Mean | SD | % RSD |
| Induction Port | 4.09 | 3.60 | 2.78 | 3.49 | 0.66 | 19 |
| Stage 1 | 15.68 | 15.24 | 13.37 | 14.76 | 1.23 | 8 |
| Stage 2 | 34.94 | 32.85 | 27.87 | 31.89 | 3.63 | 11 |
| Stage 3 | 52.70 | 52.56 | 50.62 | 51.96 | 1.16 | 2 |
| Stage 4 | 55.68 | 56.99 | 56.88 | 56.52 | 0.73 | 1 |
| Stage 5 | 30.90 | 37.66 | 39.12 | 35.89 | 4.39 | 12 |
| Stage 6 | 16.56 | 17.54 | 18.52 | 17.54 | 0.98 | 6 |
| Stage 7 | 7.06 | 7.17 | 8.78 | 7.67 | 0.97 | 13 |
| Filter | 1.91 | 3.10 | 4.76 | 3.26 | 1.43 | 44 |
| Nebulizer | 70.32 | 71.78 | 75.04 | 72.38 | 2.42 | 3 |
| (Residual Drug) | | | | | | |
| Parameter | Replicate 1 | Replicate 2 | Replicate 3 | Mean | SD | % RSD |
| MMAD (μm) | 5.20 | 4.92 | 4.62 | 4.92 | 0.29 | 6 |
| GSD | 2.04 | 2.09 | 2.09 | 2.07 | 0.03 | 1 |
| FPD <5.0 μm (mg) | 101.72 | 111.81 | 117.45 | 110.33 | 7.97 | 7 |
| FPD <3.3 μm (mg) | 56.43 | 65.46 | 71.19 | 64.36 | 7.44 | 12 |
| Fine Particle | 46.3 | 49.3 | 52.7 | 49.5 | 3.20 | 6 |
| Fraction <5.0 μm (%) | | | | | | |
| Fine Particle | 25.7 | 28.9 | 32.0 | 28.8 | 3.13 | 11 |
| Fraction <3.3 μm (%) | | | | | | |
| Delivered Dose (mg) | 219.51 | 226.70 | 222.71 | 222.97 | 3.60 | 2 |
| Amount Retained | 70.32 | 71.78 | 75.04 | 72.38 | 2.42 | 3 |
| in Nebulizer after | | | | | | |
| Dosing (Residual Drug) | | | | | | |
| Recovery (%) | 93.4 | 95.0 | 94.8 | 94.4 | 0.84 | 1 |

Stability indicating Size Exclusion Chromatography (SEC) and Reversed-Phase-HPLC (RP-HPLC) methods were developed to characterize 5 mg/mL and 50 mg/mL anakinra inhalation solutions prepared by dilution of Kineret with saline. The methods were qualified to support formulation development. Aerosol test methods were developed to characterize the 5 mg/mL and 50 mg/mL inhalation solutions when nebulized with an AeroEclipse II Jet Nebulizer. Solutions were also analyzed for pH, osmolality, surface tension, and viscosity. Finally, an activity assay was performed on the nebulized solutions.

Based on the results from this study, Kineret diluted to 5 mg/mL and 50 mg/mL, nebulized using the AeroEclipse II Jet Nebulizer and compressor, were shown to be stable to nebulization by SEC, RP-HPLC and bioactivity assays. Approximately 1 mL to 2 mL of anakinra solution remained in the nebulizer at the end of nebulization (e.g., when the nebulizer started to sputter). The concentration of the dosing solution in the nebulizer was higher post nebulization (increasing by ˜33-48% for 5 mg/mL and 50 mg/mL solutions respectively). The MMAD values of the nebulized solutions were greater than the target acceptance criteria; however it is noted that the MMAD values observed are within the 2-5 μm range considered optimal for local pulmonary delivery to the bronchioles. Tables 21 and 22 summarize the key data generated in this study.

TABLE 21
Stability of 5 and 50 mg/mL Kineret-based Anakinra Solutions Towards Nebulization Using a AeroEclipse II Jet Nebulizer and Compressor

| | Anakinra Target Solution Concentrations | | | | |
|---|---|---|---|---|---|
| | 5 mg/mL | | 50 mg/mL | | Target |
| | Initial (pre- | Post | Initial (pre- | Post | Acceptance |
| Test Attribute | nebulization) | nebulization | nebulization) | nebulization | Criteria |
| Appearance | Clear colorless | Clear colorless | Clear colorless | Clear colorless | No change in |
| | solution | solution | solution | solution | appearance from |
| | | | | | initial |
| pH | 6.36 | 6.44 | 6.30 | 6.30 | NMT +/− 0.5 pH |
| | | | | | unitsfrom initial |
| | | | | | value |
| Osmolality(mOsm/kg) | 287 | 302 | 296 | 314 | Within |
| | | | | | physiological |
| | | | | | ranges; NMT 300 |
| | | | | | mmol/L (280-295 |
| | | | | | mOsm is normal |
| | | | | | serumosmolality) |
| Surface Tension | 34.6 | | 37.8 | | Report Results |
| (mN/m) | | | | | |
| Viscosity(mPa · s) | 0.85 | | 1.06 | | Report Results |
| Inhalation | 3.998 | 5.326⁸ | 39.86 | 58.99⁸ | N/A |
| Solution Assay | | (+33.2%) | | (+48.0%) | |
| Concentration by | | | | | |
| RP-HPLC | | | | | |
| (mg/mL) | | | | | |
| Anakinra Purity % | 90.1% | 88.0% | 88.9% | 89.4% | NLT 90% of |
| byRP-HPLC | | (97.6% of initial) | | (100.6% of initial) | initial |
| Impurities by | Total: 10.0% | Total: 12.0% | Total: 11.1% | Total: 10.6% | Report all >0.1% |
| RP-HPLC | Imp ≥1.0%: | Imp ≥1.0%: | Imp ≥1.0%: | Imp ≥1.0%: | no individual >2% |
| | RRT 0.992 (1.7%), | RRT 0.953-0.958 (1.4%), | RRT 0.951(1.1%), | RRT 0.953-0.954 (1.2%), | Total <5% from |
| | RRT 1.023 (3.8%) | RRT 0.993-0.995 (1.9%), | RRT 0.992 (1.7%), | RRT 0.993-0.994 (1.6%), | initial |
| | | RRT 1.022-1.024 (3.9%) | RT 1.023 (3.8%) | RRT 1.022-1.023 (3.9%) | |
| % Monomer by | 94.8% | 96.4% | 96.1% | 96.1% | NLT 95% |
| SEC | | (101.7% of initial) | | (100.0% of initial) | monomerrelative |
| | | | | | to initial |
| % Aggregates and | Dimer: 3.2% | Dimer: 3.2% | Dimer: 3.7% | Dimer: 3.7% | NMT 5% increase in |
| fragments | Trimer: 0.1% | Trimer: 0.1% | Trimer: 0.2% | Trimer: 0.1% | aggregate/fragmen |
| | | | | | trelative to initial |

| Bioactivity, IC₅₀ | Kineret Anakinra (control) as received, IC₅₀ (μg/mL) = 0.5484 | | | | |
|---|---|---|---|---|---|
| (μg/mL) | | | | | |
| | 0.5941 | 0.5759 | 0.4546 | 0.5454 | Similar IC₅₀ values |
| | | | | | for pre- and post- |
| | | | | | nebulized samples |

TABLE 22
Aerosol Performance of 5 and 50 mg/mL Kineret-
based Anakinra Solutions Nebulized with an AeroEclipse
II Jet Nebulizer and Compressor

| | Anakinra Target | | |
|---|---|---|---|
| | Solution Concentrations | | Target Acceptance |
| Parameter | 5 mg/mL | 50 mg/mL | Criteria |
| Volume charged to | 6 | 6 | NA |
| nebulizer (mL) | | | |
| Nebulization time (min) | 10 to 14 | 10 to 14 | Between 5 and 30 |
| (time to sputter) | | | minutes |
| Drug retained in | 11.55 | 82.22 | Report results |
| nebulizerfor Dose | | | |
| Testing (mg) | | | |
| Delivered Dose (mg) | 16.06 | 238.05 | Report mean ED |
| | | | Individuals |
| | | | within +/− 35% |
| | | | of the mean ED |
| Delivered Dose % | 92.0 | 96.8 | 85-115% |
| Mass Balance | | | |
| Drug retained in | 7.99 | 72.38 | Report results |
| nebulizerfor NGI | | | |
| Testing (mg) | | | |
| NGI % Mass Balance | 92.0 | 94.4 | 85-115% |
| MMAD (μm) | 4.97 | 4.92 | NMT 4 um |
| GSD | 2.14 | 2.07 | NMT 3 |
| FPD < 5.0 μm (mg) | 9.58 | 110.33 | Report Results |
| FPD < 3.3 μm (mg) | 5.70 | 64.36 | Report Results |
| | | | Target: 10 mg |

### Example 7. Characterization of Anakinra Stability to Nebulization by Jet and Vibrating Mesh Nebulizers

Studies were performed to characterize anakinra stability to nebulization by jet and vibrating mesh nebulizers. To date two separate CMC studies have been conducted to assess the feasibility of nebulizing anakinra for pulmonary drug delivery.

### Study 1: Stability of Anakinra to Nebulization Using a Jet Nebulizer

The first study focused on the physical, chemical, and aerosol performance of an anakinra injectable product, Kineret®, prior to and after nebulization using an AeroEclipse II Jet Nebulizer and associated compressor. Kineret (anakinra) is a recombinant, non-glycosylated form of the human interleukin-1 receptor antagonist (IL-1Ra). Kineret differs from native human IL-1Ra in that it has the addition of a single methionine residue at its amino terminus. Kineret consists of 153 amino acids and has a molecular weight of 17.3 kilodaltons. It is produced by recombinant DNA technology using an e-coli bacterial expression system. Kineret (150 mg of anakinra per mL) is an injectable product provided in a pre-filled syringe containing 0.67 mL (100 mg) of anakinra in a solution (pH 6.5) containing disodium EDTA (0.12 mg), sodium chloride (5.48 mg), anhydrous citric acid (1.29 mg), and polysorbate 80 (0.70 mg) in Water for Injection, USP. The excipients in Kineret have been used in pulmonary drug products. At their current levels, the polysorbate 80 concentration exceeds the IIG listing for inhalation products (0.105 wt % vs IIG max of 0.04 wt %). A dilution to 50 mg/mL using a diluent such as saline or WFI will reduce the level of polysorbate 80 to 0.035 wt %. The initial task was to assess the viability of the current anakinra injectable formulation as a nebulized solution. As a part of this body of work, analytical methods were developed to characterize the physical, chemical, and aerosol performance of Kineret. Stability indicating SEC and RP-HPLC methods were developed to characterize 5 and 50 mg/mL anakinra inhalation solution prepared by dilution of Kineret with saline. The methods were qualified to support the formulation development. Aerosol test methods were developed to characterize the 5 and 50 mg/mL inhalation solutions when nebulized with an AeroEclipse II Jet Nebulizer. The nebulizer was operated in a continuous mode (non-breath actuated) for these studies. Solutions were also analyzed for pH, osmolality, surface tension, and viscosity. Finally, an activity assay was performed on the nebulized solutions. The developed methods were used to characterize the suitability of the formulation as a nebulized solution for delivery to the lung and evaluated against the acceptance criteria shown in Table 23.

TABLE 23
Target Acceptance Criteria for Dilutions of Kineret ® Nebulized with a Jet Nebulizer

| Test | Initial (pre-nebulization) | Post Nebulization |
|---|---|---|
| Appearance | A clear, colorless-to-white solution | No change in appearance |
| | which may contain trace amounts of | from initial |
| | small, translucent-to-white amorphous | |
| | proteinaceous particles. | |
| pH | Report initial value | NMT +/− 0.5 pH units |
| | Commercial product: | from initial value |
| | pH 6.5 +/− 0.5 | |
| | pI = 5.46 | |
| Osmolality | Report initial value | Within physiological |
| | | ranges; NMT 300 mmol/L |
| | | (280-295 mOsm is |
| | | normal serum osmolality) |
| RP-HPLC Assay | Report initial value | NLT 90% of initial |
| RP-HPLC Impurities | Report initial value | Report all >0.1% |
| | | \|no individual >2% |
| | | Total <5% |
| SEC-HPLC % monomer | Report initial value | NLT 95% monomer |
| | | relative to initial |
| SEC-HPLC % | Report initial value | NMT 5% increase in |
| aggregate/fragment | | aggregate/fragment |
| | | relative to initial |
| Bioactivity | PathHunter ® Anakinra Bioassay kit | Similar IC₅₀ values for |
| | | pre- and post-nebulized |
| | | samples |

Target Aerosol Performance Attributes

| | |
|---|---|
| Nebulization time | Between 5 and 30 minutes |
| Delivered dose | Report mean ED |
| | Individuals within +/− 35% of the mean ED |
| Drug retained in | Report results |
| nebulizer | |
| NGI % Mass balance | 85-115% |
| MMAD | NMT 4 um |
| GSD | NMT 3 |
| Fine Particle Dose | Report results |
| | Target: 10 mg FPD |

Based on the results from this study, Kineret diluted with saline to 5 and 50 mg/mL and nebulized using the AeroEclipse II Jet Nebulizer run in continuous mode were shown to be stable to nebulization by SEC, RP-HPLC and bioactivity assays. Approximately 1 to 2 mL of anakinra solution remained in the nebulizer at the end of nebulization (e.g., when the nebulizer started to sputter). The concentration of the anakinra solution remaining in the nebulizer was higher post-nebulization than the pre-nebulization solution concentration (increasing by ˜33% and ˜48% for 5 and 50 mg/mL solutions respectively). The MMAD of the nebulized solutions were greater than the target acceptance criteria of 4 μm; however, it is noted that the MMAD values observed are within the 2-5 μm range considered optimal for local pulmonary delivery to the bronchioles. See Table 12 (above) for a summary of the results.

Although anakinra nebulized using a jet nebulizer was shown to be stable, due to the recirculation that occurs in jet nebulizer during dosing, the protein solution retained in the device concentrated over time. This can impact the uniformity of the delivered dose over the nebulization duration. Since vibrating mesh nebulizers are known to reduce the volume of solution left in the nebulizer post dose, and they do not have the same recirculation issues associated with the jet nebulizers, the team decided to evaluate a vibrating mesh nebulizer in the second study. In addition to evaluating a different nebulizer, the team also evaluated a second source of anakinra.

### Study 2: Characterization of Anakinra Nebulized by a Vibrating Mesh Nebulizer for Two Different Protein Sources

This study evaluated the chemical, physical, and aerosol properties of two anakinra protein sources using a Philips InnoSpire Go vibrating mesh nebulizer. Anakinra sourced from Paras was compared to the Sobi Kineret® supply used in the first study. Both anakinra formulation matrices and initial concentrations were the same (e.g., both formulations used the Kineret formulation matrix and concentration). Both anakinra starting formulations were diluted to 50 mg/mL using saline for all testing which included appearance, pH, osmolality, viscosity, surface tension, activity, delivered dose and aerodynamic particle size distribution. Select aerosol samples were also analyzed for impurities by both HPLC-RP and HPLC-SEC before and after nebulization. The same acceptance criteria used in the first study was applied to this study with the addition of attributes for surface tension and viscosity.

Based on the study results, anakinra was shown to be stable to nebulization using a vibrating mesh nebulizer. The physical testing was performed on the 50 mg/mL inhalation solutions prior to nebulization. Appearance testing demonstrated all solutions were clear, colorless, and free of any foreign particulate matter. The pH of the solutions was consistent at 6.3. The osmolality of the inhalation solutions were close to isotonic at 296 and 303 mOsm/kg for the Sobi and Paras formulations, respectively. The surface tension for the inhalation solution were 37.8 and 32.8 mN/m for the Sobi and Paras formulations, respectively. The viscosity for the inhalation solutions were similar.

A difference in chemical purity was noted between the two anakinra protein supplies. The purity of the 50 mg/mL Sobi anakinra inhalation solution prior to nebulization was 96% monomer by HPLC-SEC and 90% by area by RP-HPLC. The purity of the 50 mg/mL Paras inhalation solution prior to nebulization was 99% monomer by HPLC-SEC and 73% by area by RP-HPLC. Purity of the delivered dose samples did not change significantly post nebulization for either protein supply.

Nebulization time for 6 mL of the 50 mg/mL anakinra solutions ranged from 10-14 minutes and only a small amount of anakinra solution was left in the nebulizers post nebulization. The aerodynamic properties for both anakinra sources were similar and the MMAD values did not meet the acceptance criteria (˜4.7 μm compared with target of NMT 4 μm). However, it is noted that at 4.7 μm, the MMAD value observed is within the 2-5 μm range considered optimal for local pulmonary delivery to the bronchioles. See Table 7 (above) for a summary of the results.

Based on the results from these 2 studies, anakinra can be nebulized by either a jet or vibrating mesh nebulizer. This outcome was also confirmed for both sources of anakinra; even though the Paras supply was less pure by RP-HPLC. Paras has indicated that the source of the impurity was the polysorbate 80 used and they have identified an injectable grade of the excipient that will address the purity issues and a resupply of higher purity protein will be provided shortly.

As noted, even though the MMAD values of the diluted formulations in both studies and by both nebulizers did not meet the target acceptance criteria of no more than 4 μm, all MMAD values obtained were within the acceptable range of 2-5 μm which is considered acceptable for local pulmonary delivery to the bronchioles. Based on the formulation composition of the Kineret product, removal and/or adjustment of existing formulation excipient levels to increase the surface tension and/or an increase in viscosity is expected to result in a lower MMAD value Phase 1. A breath actuated vibrating mesh nebulizer is also desirable to reduce losses during nebulization. Breathing patterns were not used in these studies (USP <1601>) and will be implemented in future studies. The AeroEclipse II Jet nebulizer was used in continuous mode and will be evaluated using breathe actuation to obtain a better estimate of the lung dose being delivered from this nebulizer. Table 24 summarizes the combined results from studies 1 and 2 described herein.

TABLE 24
Combined Results from Studies 1 and 2

| | STUDY 1 | | STUDY 2 | |
|---|---|---|---|---|
| Nebulizer | AeroEclipse II | | InnoSpire Go | |
| API Source | Sobi | Sobi | Sobi | Paras |
| Inhalation Solution | 5 mg/mL | 50 mg/mL | 50 mg/mL | 50 mgl/mL |
| Concentration | | | | |
| Nebulizer Charge | 6 | 6 | 6 | 6 |
| Volume (mL) | | | | |
| Appearance | Clear | Clear | Clear | Clear |
| | colorless | colorless | colorless | colorless |
| | solution | solution | solution | solution |
| Physicochemical | | | | |
| Properties | | | | |
| pH | 6.36 | 6.30 | 6.3 | 6.3 |
| Osmolality (mOsm/kg) | 287 | 296 | 296 | 303 |
| Surface Tension (mN/m) | 34.6 | 37.8 | 37.8 | 32.8 |
| Density (g/mL) | 1.05 | 1.05 | 1.05 | 1.05 |
| Viscosity (mPa · s) | 0.85 | 1.06 | 1.061 | 1.144 |
| Aerosol Performance | | | | |
| Delivered Dose Results | | | | |
| (n = 5) | | | | |
| Actual Solution | 5.28 | 58.56 | NR | NR |
| Concentration (mg/mL) | | | | |
| Actual Nebulizer Charge | 30.00 | 339.83 | 275.12 | 281.90 |
| (mg) | | | | |
| Delivered Dose (mg) | 16.06 | 238.05 | 248.55 | 252.05 |
| DD (% of charge dose) | 53.5% | 70.0% | 90.3% | 89.4% |
| Estimated Residual | 2.31 | 1.40 | 0.61 | 0.49 |
| Volume in Nebulizer (mL) | | | | |
| APSD by NGI Data (n = 3) | | | | |
| MMAD (μm) | 4.97 | 4.92 | 4.75 | 4.70 |
| GSD | 2.14 | 2.07 | 1.75 | 1.79 |
| FPD < 5.0 μm (mg) | 9.58 | 110.33 | 121.86 | 131.01 |
| FPD < 3.3 μm (mg) | 5.70 | 64.36 | 59.82 | 66.63 |
| Fine Particle Fraction < | 48.8 | 49.5 | 52.2 | 52.9 |
| 5.0 μm (%) | | | | |
| Fine Particle Fraction < | 29.0 | 28.8 | 25.6 | 27.0 |
| 3.3 μm (%) | | | | |
| Delivered Dose by NGI (mg) | 19.63 | 222.71 | 232.87 | 247.23 |
| Purity by RP-HPLC | | | | |
| (% anakinra) | | | | |
| Pre Nebulization | 90.1% | 88.9% | 90.0% | 73.3% |
| Post Nebulization | 88.0% | 89.4% | 89.9% | 73.9% |
| (dose sample) | | | | |
| Post Nebulization | — | — | 88.1% | 72.5% |
| (retained sample) | | | | |
| Change in Solution | 33.2% | 48.0% | — | — |
| Concentration Pre- | | | | |
| Nebulization vs Post- | | | | |
| Nebulization Residual | | | | |
| Solution Concentration | | | | |
| (% change) | | | | |
| Purity by SEC | | | | |
| (% anakinra monomer) | | | | |
| Pre-Nebulization | 94.8% | 96.1% | 95.60 | 99.30 |
| Post Nebulization | 96.4% | 96.1% | 95.70 | 99.30 |
| (dose sample) | | | | |
| Post Nebulization | | | 96.10 | 99.60 |
| (retained sample) | | | | |
| Bioactivity IC₅₀ | — | — | Kineret Anakinra, | Paras Anakinra, |
| Values (μg/mL) | | | as received | as received |
| | | | IC₅₀ = 0.5966 | IC₅₀ = 0.3765 |
| Pre-Nebulized (Control) | 0.5941 | 0.4546 | 0.3865 | 0.23 |
| Nebulized Dose | 1.095 | 0.5938 | 0.5406 | 0.4439 |
| | — | — | 0.5688 | 0.5458 |
| | — | — | 0.5675 | 0.6 |
| Residual Dose in Nebulizer | 0.6457 | 0.4539 | 0.5947 | 0.489 |
| | — | — | 0.8023 | 0.6057 |
| Post-Nebulized (Control) | 0.5759 | 0.5454 | — | — |

1. A method for treating an inflammatory disorder of the lower airways in a human subject in need thereof, comprising administering an effective amount of a recombinant human IL-1 receptor antagonist (rhIL-1Ra) directly to the lower airways in the human subject, wherein the inflammatory disorder is caused by a coronavirus infection.

2. The method of claim 1, wherein the rhIL-1Ra is anakinra.

3. The method of claim 2, wherein the anakinra is a component of a composition, and wherein the composition is an inhaled formulation.

4. The method of claim 3, wherein the inhaled formulation is ALTA-2530.

5. The method of claim 1, wherein the coronavirus infection is caused by a coronavirus selected from the group consisting of SARS-CoV-2, SARS-CoV, MERS-CoV, 229E, NL63, OC43, and HKU1.

6. The method of claim 1, wherein the coronavirus infection is caused by a coronavirus selected from the group consisting of SARS-CoV-2 and a mutant thereof.

7. The method of claim 6, wherein the SARS-CoV-2 mutant is a variant selected from the group consisting of B.1.526, B.1.526.1, B.1.525, B.1.617, B.1.617.1, B.1.617.2, B.1.617.3, B.1.1.7, B.1.351, B.1.427, B.1.429, P.1, and P.2.

8. The method of claim 1, wherein the human subject is diagnosed with COVID-19.

9. The method of claim 1, wherein the inflammatory disorder of the lower airways is acute respiratory distress syndrome or cytokine storm syndrome.

10. The method of claim 1, wherein the rhIL-1Ra is nebulized.

11. The method of claim 10, wherein the nebulized rhIL-1Ra has a mass median aerodynamic diameter (MMAD) of about 1 μm to 15 μm.

12. The method of claim 11, wherein the nebulized rhIL-1Ra has a mass median aerodynamic diameter (MMAD) of about 3 μm.

13. The method of claim 10, wherein the nebulized rhIL-1Ra is delivered using a nebulizer.

14. The method of claim 13, wherein the nebulizer is selected from the group consisting of PARI eFlow nebulizer, PARI VELOX nebulizer, Philips iNeb Advanced nebulizer, Philips InnoSpire Go nebulizer, a Vectura nebulizer, and AeroEclipse II nebulizer.

15. The method of claim 14, wherein the nebulizer is a PARI nebulizer or a Vectura nebulizer.

16. The method of claim 1, wherein the rhIL-1Ra inhibits at least one pro-inflammatory cytokine selected from the group consisting of interleukin 1 alpha (IL-1α), interleukin 1 beta (IL-1β), interleukin 6 (IL-6), tumor necrosis factor alpha (TNFα), and interleukin 18 (IL-18).

17. A method for treating an inflammatory disorder of the lower airways in a human subject in need thereof, comprising administering an effective amount of a recombinant human IL-1 receptor antagonist (rhIL-1Ra) directly to the lower airways in the human subject, wherein the rhIL-1Ra causes blockade of interleukin 1 to about the same degree as caused by the upregulation of endogenous IL-1Ra during a restoration of physiologic immune regulation.

18. The method of claim 17, wherein the rhIL-1Ra is anakinra.

19. The method of claim 17, wherein the anakinra is a component of a composition, and wherein the composition is an inhaled formulation.

20. The method of claim 19, wherein the inhaled formulation is ALTA-2530.

21. The method of claim 17, wherein the inflammatory disorder is caused by a coronavirus infection.

22. The method of claim 17, wherein the human subject is diagnosed with a coronavirus infection.

23. The method of claim 22, wherein the coronavirus infection is COVID-19.

24. The method of claim 21, wherein the coronavirus infection is caused by a coronavirus selected from the group consisting of SARS-CoV-2, SARS-CoV, MERS-CoV, 229E, NL63, OC43, and HKU1.

25. The method of claim 24, wherein the coronavirus infection is caused by a coronavirus selected from the group consisting of SARS-CoV-2 and a mutant thereof.

26. The method of claim 25, wherein the SARS-CoV-2 mutant is a variant selected from the group consisting of B.1.526, B.1.526.1, B.1.525, B.1.617, B.1.617.1, B.1.617.2, B.1.617.3, B.1.1.7, B.1.351, B.1.427, B.1.429, P.1, and P.2.

27. The method of claim 17, wherein the inflammatory disorder of the lower airways is acute respiratory distress syndrome or cytokine storm syndrome.

28. The method of claim 17, wherein the rhIL-1Ra is nebulized.

29. The method of claim 28, wherein the nebulized rhIL-1Ra has a mass median aerodynamic diameter (MMAD) of about 1 μm to 15 μm.

30. The method of claim 29, wherein the nebulized rhIL-1Ra has a mass median aerodynamic diameter (MMAD) of about 3 μm.

31. The method of claim 28, wherein the nebulized rhIL-1Ra is delivered using a nebulizer.

32. The method of claim 31, wherein the nebulizer is selected from the group consisting of PARI eFlow nebulizer, PARI VELOX nebulizer, Philips iNeb Advanced nebulizer, Philips InnoSpire Go nebulizer, a Vectura nebulizer, and AeroEclipse II.

33. The method of claim 32, wherein the nebulizer is a PARI nebulizer or a Vectura nebulizer.

34. The method of claim 17, wherein the rhIL-1Ra inhibits at least one pro-inflammatory cytokine selected from the group consisting of interleukin 1 alpha (IL-1α), interleukin 1 beta (IL-1β), interleukin 6 (IL-6), tumor necrosis factor alpha (TNFα), and interleukin 18 (IL-18).
